SEQUENCE LISTING (46 sequences)

SEQ ID NO: 1 - protein, 10 aa, synthetic construct
GDSVSSNSAT

SEQ ID NO: 2 - protein, 9 aa, synthetic construct
TYYRSKWYN

SEQ ID NO: 3 - protein, 13 aa, synthetic construct
ARGMMTYYYGMDV

SEQ ID NO: 4 - protein, 9 aa, synthetic construct
SGINVGPYR

SEQ ID NO: 5 - protein, 7 aa, synthetic construct
YKSDSDK

SEQ ID NO: 6 - protein, 9 aa, synthetic construct
MIWHSSAAV

SEQ ID NO: 7 - protein, 128 aa, synthetic construct
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGS

SEQ ID NO: 8 - protein, 116 aa, synthetic construct
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 9 - protein, 259 aa, synthetic construct
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 10 - protein, 15 aa, synthetic construct
GGGGSGGGGSGGGGS

SEQ ID NO: 11 - protein, 45 aa, synthetic construct
TTTPAPRPPTPAPTIASQPLSLRPEACRPAAGGAVHTRGLDFACD

SEQ ID NO: 12 - protein, 24 aa, synthetic construct
IYIWAPLAGTCGVLLLSLVITLYC

SEQ ID NO: 13 - protein, 42 aa, synthetic construct
KRGRKKLLYIFKQPFMRPVQTTQEEDGCSCRFPEEEEGGCEL

SEQ ID NO: 14 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 19 aa, synthetic construct
MDWTWRILFLVAAATGAHS

SEQ ID NO: 16 - protein, 504 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 1512 bp, synthetic construct
  source 1..1512: mol_type = other DNA
atggactggacctggaggatcctgtttctggtggccgccgccacaggagcccacagccaggtgcagctgcagcagagcggacctggcctggtgacacccagccagaccctgagcctgacctgtgccatctccggcgatagcgtgagcagcaacagcgccacctggaactggatcaggcagagccccagcagaggactggagtggctgggcaggacctactacaggagcaagtggtacaacgactacgccgtgagcgtgaagagcaggatgagcatcaaccccgacaccagcaagaaccagttctccctgcagctgaactccgtgacccccgaggacaccgccgtgtactactgcgccaggggcatgatgacctactactacggcatggacgtgtggggccagggaaccaccgtgaccgtgagcagcggcatcctgggcagcggcggcggcggcagcggcggcggcggcagcggaggaggcggaagccagcctgtgctgacccagagcagcagcctgagcgctagccctggagctagcgccagcctgacctgcaccctgagaagcggcatcaacgtgggcccctacaggatctactggtaccagcagaagcctggcagccccccccagtacctgctgaactacaagagcgacagcgacaagcagcagggcagcggcgtgcctagcagattcagcggcagcaaggatgccagcgccaacgccggagtgctgctgatcagcggcctgaggagcgaggatgaggccgactactactgcatgatctggcacagcagcgccgccgtgtttggaggcggaacccagctgaccgtgctgagcgcggccgcaaccaccacccccgcccctagacctcctacacccgctcccacaatcgccagccagcctctgtctttaagacccgaggcttgtagacccgctgctggcggcgccgtgcataccagaggactggacttcgcttgtgacatctacatctgggctcctttagccggcacatgtggagtgctgctgctgtctttagtgatcactttatactgcaagaggggtcgtaagaagctgctgtacatcttcaagcagcccttcatgaggcccgtgcagaccacccaagaagaggacggctgcagctgtcgttttcccgaagaggaggagggcggctgcgagctgagggtgaagttcagcagaagcgccgatgcccccgcttaccagcaaggtcagaaccagctgtacaacgagctgaatttaggtcgtagggaggagtacgacgtgctggacaagaggaggggcagagaccccgaaatgggcggcaagcctcgtaggaagaacccccaagaaggtttatacaacgagctgcagaaggacaagatggccgaggcctacagcgagatcggcatgaagggcgagaggaggagaggcaagggccacgacggtttataccaaggtctgagcaccgccaccaaggacacctacgatgctttacacatgcaagctttacctcctcgt

SEQ ID NO: 18 - protein, 177 aa, unidentified
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 98 aa, unidentified
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 20 - protein, 26 aa, synthetic construct
SGGGSGGGGSGGGGSGGGGSGGGSLQ

SEQ ID NO: 21 - protein, 20 aa, synthetic construct
SGGSGGGGSGGGSGGGGSLQ

SEQ ID NO: 22 - protein, 17 aa, synthetic construct
GSGGGGSGGGGSGGGGS

SEQ ID NO: 23 - protein, 22 aa, synthetic construct
GSGATNFSLLKQAGDVEENPGP

SEQ ID NO: 24 - DNA, 470 bp, synthetic construct
  source 1..470: mol_type = other DNA
ctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 25 - DNA, 2472 bp, synthetic construct
  source 1..2472: mol_type = other DNA
atggactggacctggaggatcctgtttctggtggccgccgccacaggagcccacagccaggtgcagctgcagcagagcggacctggcctggtgacacccagccagaccctgagcctgacctgtgccatctccggcgatagcgtgagcagcaacagcgccacctggaactggatcaggcagagccccagcagaggactggagtggctgggcaggacctactacaggagcaagtggtacaacgactacgccgtgagcgtgaagagcaggatgagcatcaaccccgacaccagcaagaaccagttctccctgcagctgaactccgtgacccccgaggacaccgccgtgtactactgcgccaggggcatgatgacctactactacggcatggacgtgtggggccagggaaccaccgtgaccgtgagcagcggcatcctgggcagcggcggcggcggcagcggcggcggcggcagcggaggaggcggaagccagcctgtgctgacccagagcagcagcctgagcgctagccctggagctagcgccagcctgacctgcaccctgagaagcggcatcaacgtgggcccctacaggatctactggtaccagcagaagcctggcagccccccccagtacctgctgaactacaagagcgacagcgacaagcagcagggcagcggcgtgcctagcagattcagcggcagcaaggatgccagcgccaacgccggagtgctgctgatcagcggcctgaggagcgaggatgaggccgactactactgcatgatctggcacagcagcgccgccgtgtttggaggcggaacccagctgaccgtgctgagcgcggccgcaaccaccacccccgcccctagacctcctacacccgctcccacaatcgccagccagcctctgtctttaagacccgaggcttgtagacccgctgctggcggcgccgtgcataccagaggactggacttcgcttgtgacatctacatctgggctcctttagccggcacatgtggagtgctgctgctgtctttagtgatcactttatactgcaagaggggtcgtaagaagctgctgtacatcttcaagcagcccttcatgaggcccgtgcagaccacccaagaagaggacggctgcagctgtcgttttcccgaagaggaggagggcggctgcgagctgagggtgaagttcagcagaagcgccgatgcccccgcttaccagcaaggtcagaaccagctgtacaacgagctgaatttaggtcgtagggaggagtacgacgtgctggacaagaggaggggcagagaccccgaaatgggcggcaagcctcgtaggaagaacccccaagaaggtttatacaacgagctgcagaaggacaagatggccgaggcctacagcgagatcggcatgaagggcgagaggaggagaggcaagggccacgacggtttataccaaggtctgagcaccgccaccaaggacacctacgatgctttacacatgcaagctttacctcctcgtggaagcggagctactaacttcagcctgctgaagcaggctggagacgtggaggagaaccctggaccctgcatgttccatgtgagcttcaggtacatcttcggactgcctcctctcatcctggtcctcctccccgtggccagctccgactgtgacatcgaaggaaaggatggcaagcagtacgaaagcgtgctgatggtgagcatcgatcagctcctggattccatgaaggaaatcggctccaactgcctcaacaatgagttcaacttttttaagaggcatatctgcgacgccaacaaggagggcatgtttctgttcagggccgccaggaagctgagacagttcctcaagatgaatagcaccggcgacttcgacctccatctgctgaaggtgtccgagggaaccaccatcctgctgaactgcaccggccaagtgaagggaagaaaacctgctgccctgggcgaggctcagcctaccaagagcctggaggagaacaaaagcctgaaggagcagaagaagctgaacgacctgtgcttcctcaagaggctcctgcaggagattaagacctgttggaacaagatcctgatgggcacaaaggagcacggcagcggcgccaccaacttctctctgctgaagcaagccggcgatgtggaggagaatcccggccccatggctctgctgctcgccctgagcctgctcgtcctctggacctcccctgctcctaccctgagcggcaccaatgacgctgaagactgctgcctgtccgtgacccagaagcctatccccggatatatcgtgaggaattttcattacctcctgatcaaggacggctgtagagtgcccgccgtcgtgttcacaacactcagaggcaggcagctgtgtgctccccccgaccagccttgggtggagagaatcattcagagactgcaaaggacctccgctaagatgaagaggaggtccagc

SEQ ID NO: 26 - DNA, 66 bp, Teschovirus A
  source 1..66: mol_type = genomic DNA
ggaagcggagctactaacttcagcctgctgaagcaggctggagacgtggaggagaaccctggaccc

SEQ ID NO: 27 - DNA, 66 bp, synthetic construct
  source 1..66: mol_type = other DNA
ggcagcggcgccaccaacttctctctgctgaagcaagccggcgatgtggaggagaatcccggcccc

SEQ ID NO: 28 - protein, 25 aa, synthetic construct
IYIWAPLAGTCGVLLLSLVITLYCN

SEQ ID NO: 29 - protein, 46 aa, synthetic construct
PTTTPAPRPPTPAPTIASQPLSLRPEACRPAAGGAVHTRGLDFACD

SEQ ID NO: 30 - protein, 4 aa, synthetic construct
RAAA

SEQ ID NO: 31 - protein, 505 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 501 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 502 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 502 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 503 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 506 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 505 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAAATTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 505 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAAAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 506 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 506 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAATTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 507 aa, synthetic construct
MDWTWRILFLVAAATGAHSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 19 aa, Teschovirus A
ATNFSLLKQAGDVEENPGP

SEQ ID NO: 43 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 44 - protein, 20 aa, Equine rhinitis A virus
QCTNYALLKLAGDVESNPGP

SEQ ID NO: 45 - protein, 22 aa, Foot-and-mouth disease virus
VKQTLNFDLLKLAGDVESNPGP

SEQ ID NO: 46 - protein, 18 aa, Thosea asigna virus
EGRGSLLTCGDVEENPGP

(19) United States
(12) Patent Application Publication
(10) Pub. No.: US 2024/0350627 A1
(43) Pub. Date: Oct. 24, 2024

(54) ENGINEERED IMMUNE CELL THAT SPECIFICALLY TARGETS MESOTHELIN AND USES THEREOF

(71) Applicant: Takeda Pharmaceutical Company Limited, Chuo-ku, Osaka-shi, Osaka (JP)

(72) Inventors: Chihiro Take, Kanagawa (JP); Akiko Yamaguchi, Kanagawa (JP); Gary Shapiro, Cambridge, MA (US)

(73) Assignee: Takeda Pharmaceutical Company Limited, Chuo-ku, Osaka-shi, Osaka (JP)

(21) Appl. No.: 18/292,586

(22) PCT Filed: Jul. 28, 2022
(86) PCT No.: PCT/IB2022/057006

§ 371 Date: Jan. 26, 2024

Related U.S. Application Data
(60) Provisional application No. 63/227,116, filed on Jul. 29, 2021.

(51) Int. Cl. A61K 39/00; A61P 35/00; C07K 14/52; C07K 14/54; C07K 14/705; C07K 14/725; C07K 16/30; C12N 5/0783

(52) U.S. Cl. CPC A61K 39/4611; A61K 39/4631; A61K 39/4635; A61K 39/464468; A61P 35/00; C07K 14/521; C07K 14/5418; C07K 14/7051; C07K 14/70517; C07K 14/70578; C07K 16/30; C12N 5/0636; C07K 2317/53; C07K 2317/622; C07K 2319/02; C07K 2319/03; C12N 2510/00

(57) ABSTRACT

Disclosed herein are isolated nucleic acid molecules comprising a polynucleotide encoding a chimeric antigen receptor (CAR) comprising an antibody that specifically recognizes human mesothelin, a CD8 hinge region, a CD8 transmembrane region, a 4-1BB intracellular region and a CD3ζ intracellular region; a polynucleotide encoding IL-7; and a polynucleotide encoding CCL19. Also disclosed herein include vectors, modified immune cells, and pharmaceutical compositions comprising the nucleic acid molecules and methods of use.

### CROSS-REFERENCE TO RELATED APPLICATIONS

This application is the U.S. National Stage of PCT/IB2022/057006, filed Jul. 28, 2022, which claims priority under 35 U.S.C § 119(e) to U.S. Provisional Application No. 63/227,116, filed Jul. 29, 2021, which is hereby incorporated by reference in its entirety.

The instant application contains a Sequence Listing which has been submitted in XML format and is hereby incorporated by reference in its entirety. Said XML copy, created on Jan. 24, 2024, is named 087147-0693_SL.xml and is 52,920 bytes.

### TECHNICAL FIELD

The present invention relates to an immune cell that expresses a cell surface molecule specifically recognizing human mesothelin, interleukin 7 (IL-7), and chemokine (C—C motif) ligand 19 (CCL19), a pharmaceutical composition comprising the immune cell, an expression vector comprising a polynucleotide encoding a cell surface molecule specifically recognizing mesothelin, a polynucleotide encoding IL-7, and a polynucleotide encoding CCL19, a method of use, and a method for producing an immune cell that expresses a cell surface molecule specifically recognizing human mesothelin, IL-7, and CCL19, comprising introducing a polynucleotide encoding the cell surface molecule specifically recognizing human mesothelin, a polynucleotide encoding the IL-7, and a polynucleotide encoding the CCL19 to an immune cell.

### BACKGROUND OF THE DISCLOSURE

Malignant tumors are diseases that affect many people in the world and in general, are widely treated by chemotherapy, radiotherapy, or surgical therapy. However, there have been various problems such as the occurrence of adverse reactions, a loss of some functions, and recurrence or metastasis which cannot be treated. As such, the development of immune cell therapy has been advanced in recent years in order to maintain higher quality of life (QOL) of patients. Immune cell therapy involves harvesting immune cells from a patient, performing procedures to enhance the immune functions of the harvested immune cells, amplifying the cells, and readministering the cells back to the patient. For example, the immune cell therapy can include collecting T cells from a patient, introducing a nucleic acid encoding chimeric antigen receptor (constitutive androstane receptor: hereinafter, also referred to as “CAR”) to the T cells, and readministering the T cells back to the patient. Although early success in blood cancers have been observed with CAR-T therapies; life-threatening toxicities and a substantial lack of efficacy in the treatment of solid tumors have also been observed. As such, improved CAR-T therapies are needed.

### PRIOR ART DOCUMENTS

### Patent Documents

Patent Document 1: WO2016/056228
Patent Document 2: WO2019/124468
Patent Document 3: WO2013/063419

### Non-Patent Documents

Non-patent Document 1: Adachi, et al., “IL-7 and CCL19 expression in CAR-T cells improves immune cell infiltration and CAR-T cell survival in the tumor,” Nature Biotech, 36(4): 346-353, 2018.

### SUMMARY OF THE DISCLOSURE

### Objective Technical Problem to be Solved by the Invention

There are a number of challenges that exist with immunotherapy with T cells, such as insufficient trafficking to solid tumors, high toxicity to normal tissues, the inability to overcome the immunosuppressive tumor microenvironment, and insufficient activation of the endogenous immune response. Further, immune cells modified to express CARs that specifically recognizes mesothelin have been shown to exhibit minimal therapeutic efficacy. Therefore, an objective technical problem to be solved is to provide optimized modified immune cells to target mesothelin-expressing cancers.

### Means to Solve the Objective Technical Problem

The present inventors have discovered that immune cells modified to express a CAR that specifically recognizes mesothelin, IL-7, and CCL19 can improve therapeutic efficacy of the immunotherapy and to improve survival rate.

In certain embodiments, the present invention comprises an isolated nucleic acid molecule comprising: a polynucleotide encoding a chimeric antigen receptor (CAR) comprising an antibody that specifically recognizes human mesothelin, a CD8 hinge region, a CD8 transmembrane region, a 4-1BB intracellular region, and a CD3ζ intracellular region; a polynucleotide encoding IL-7; and a polynucleotide encoding CCL19. In some embodiments, the IL-7 is human IL-7. In some embodiments, the CCL19 is human CCL19. In some embodiments, the antibody comprises a heavy chain variable region (VH) and a light chain variable region (VL), wherein the VH comprises three complementarity-determining regions (CDRs) comprising SEQ ID NOs: 1-3, and wherein the VL comprises three CDRs comprising SEQ ID NOs: 4-6. In some embodiments, the VH comprises SEQ ID NO: 7 and the VL comprises SEQ ID NO: 8. In some embodiments, the antibody comprises a single-chain variable fragment (scFv) format. In some embodiments, the antibody comprises SEQ ID NO: 9. In some embodiments, the 4-1BB intracellular region comprises of SEQ ID NO: 13. In some embodiments, the CD3ζ intracellular region comprises SEQ ID NO: 14. In some embodiments, the 4-1BB intracellular region is upstream of the CD3ζ intracellular region in the isolated nucleic acid molecule. In some embodiments, the CD8 hinge region comprises SEQ ID NO: 11. In some embodiments, the CD8 transmembrane region comprises SEQ ID NO: 12. In some embodiments, the nucleic acid further comprises a peptide linker 3 to 10 amino acid residues in length linking the antibody and the CD8 hinge region. In some embodiments, the peptide linker comprises AAA. In some embodiments, the isolated nucleic acid molecule further comprises a signaling peptide. In some embodiments, the signaling peptide is located upstream of the antibody that specifically recognizes human mesothelin in the isolated nucleic acid molecule. In some embodiments, the signaling peptide comprises SEQ ID NO: 15. In some embodiments, the polynucleotide encoding IL-7 and the polynucleotide encoding CCL19 are each independently transcribed under a promoter comprising a polynucleotide encoding a self-cleaving 2A peptide (2A peptide). In some embodiments, the 2A peptide is P2A, optionally comprising ATNFSLLKQAGDVEENPGP (SEQ ID NO: 42). In some embodiments, a peptide linker is further added to the N-terminus of the 2A peptide, wherein the peptide linker comprises GSG. In some embodiments, the IL-7 comprises SEQ ID NO: 18. In some embodiments, the CCL19 comprises SEQ ID NO: 19. In some embodiments, the polynucleotide encoding the CAR, the polynucleotide encoding IL-7, and the polynucleotide encoding CCL19 are arranged in the nucleic acid molecule from the 5′ terminus to the 3′ terminus as the polynucleotide encoding the CAR—the polynucleotide encoding IL-7—the polynucleotide encoding CCL19. In some embodiments, the isolated nucleic acid molecule encodes a polypeptide comprising SEQ ID NO: 16. In some embodiments, the isolated nucleic acid molecule comprises SEQ ID NO: 17. In some embodiments, the isolated nucleic acid molecule comprises SEQ ID NO: 25.

In certain embodiments, the present invention comprises a vector comprising the nucleic acid molecule described herein. In some embodiments, the vector is a viral vector, optionally an expression vector. In some embodiments, the viral vector is selected from a retrovirus vector, a lentivirus vector, an adenovirus vector, and an adeno-associated virus (AAV) vector. In some embodiments, the viral vector is gamma retrovirus vector. In some embodiments, the viral vector is a pSFG vector, a pMSGV vector, or a pMSCV vector. In some embodiments, the vector is a plasmid.

In certain embodiments, the present invention comprises an immune cell derived from a mammal or separated from a mammal and comprising the nucleic acid molecule described herein or the vector described herein. In some embodiments, the present invention comprises an immune cell derived from a mammal or separated from a mammal and expressing a) a chimeric antigen receptor (CAR) comprising an antibody that specifically recognizes human mesothelin, a CD8 hinge region, a CD8 transmembrane region, a 4-1BB intracellular region and a CD3ζ intracellular region, b) IL-7, and c) CCL19. In some embodiments, the immune cell is a T cell, a natural killer (NK) cell, a B cell, an antigen presenting cell, or a granulocyte, optionally a T cell or an NK cell.

In certain embodiments, the present invention comprises a pharmaceutical composition comprising the immune cell described herein, and a pharmaceutically acceptable additive.

In certain embodiments, the present invention comprises a method of treating a mesothelin-expressing cancer comprising administering to a subject in need thereof the immune cell described herein or the pharmaceutical composition described herein. In some embodiments, the mesothelin-expressing cancer is a solid tumor, optionally selected from mesothelioma, colorectal cancer, pancreatic cancer, thymic cancer, bile duct cancer, lung cancer, skin cancer, breast cancer, prostate cancer, urinary bladder cancer, virginal cancer, neck cancer, uterine cancer, liver cancer, kidney cancer, spleen cancer, tracheal cancer, bronchial cancer, stomach cancer, esophageal cancer, gallbladder cancer, testis cancer, ovarian cancer, and bone cancer. In some embodiments, the mesothelin-expressing cancer is a hematopoietic cancer. In some embodiments, the mesothelin-expressing cancer is a sarcoma, optionally selected from chondrosarcoma, Ewing's sarcoma, malignant hemangioendothelioma, malignant schwannoma, osteosarcoma, and soft tissue sarcoma. In some embodiments, the mesothelin-expressing cancer is a metastatic cancer. In some embodiments, the mesothelin-expressing cancer is a relapsed cancer or a refractory cancer. In some embodiments, the method further comprises administering to the subject an additional therapeutic agent or an additional therapeutic regimen. In some embodiments, the additional therapeutic agent comprises a chemotherapeutic agent, an immunotherapeutic agent, a targeted therapy, radiation therapy, or a combination thereof. In some embodiments, the additional therapeutic regimen comprises a first-line therapy. In some embodiments, the additional therapeutic regimen comprises surgery. In some embodiments, the immune cell described above or the pharmaceutical composition described above and the additional therapeutic agent are administered simultaneously. In some embodiments, the immune cell described above or the pharmaceutical composition described above and the additional therapeutic agent are administered sequentially. In some embodiments, the immune cell described above or the pharmaceutical composition described above is administered to the subject prior to administration of the additional therapeutic agent. In some embodiments, the immune cell described above or the pharmaceutical composition described above is administered to the subject after administration of the additional therapeutic agent. In some embodiments, the subject is a human.

In certain embodiments, the present invention comprises a method of decreasing tumor cell proliferation comprising contacting the tumor cell with the immune cell described herein, thereby decreasing the tumor cell proliferation. In some embodiments, the method is an in vitro method. In some embodiments, the method is an in vivo method.

In certain embodiments, the present invention comprises a method for producing an immune cell expressing cell surface molecules that specifically recognize human mesothelin, IL-7, and CCL19, the method comprising: introducing the nucleic acid molecule described herein or the vector described herein to an immune cell to induce expression of cell surface molecules that specifically recognize human mesothelin, IL-7, and CCL19 by the immune cell. In some embodiments, the immune cell is a T cell, a natural killer (NK) cell, a B cell, an antigen presenting cell, or a granulocyte, optionally a T cell or an NK cell.

In certain embodiments, the present invention comprises a kit comprising the nucleic acid molecule described herein; the vector described herein, the immune cell described herein, or the pharmaceutical composition described herein, and instructions of use.

### Effect of the Invention

The immune cell of the present invention has cytotoxic activity against cancer cells expressing mesothelin (e.g., human mesothelin) and is capable of suppressing the formation of tumor expressing mesothelin (e.g., human mesothelin). Also, the immune cell of the present invention has suppressive effects on the recurrence of cancer cells. Also, the immune cell of the present invention has superior safety profile.

### BRIEF DESCRIPTION OF THE DRAWINGS

FIG. 1A shows a cartoon representation of an exemplary vector comprising a polynucleotide encoding a chimeric antigen receptor (CAR) that specifically recognizes mesothelin, a polynucleotide encoding IL-7, and a polynucleotide encoding CCL19.

FIG. 1B shows a cartoon representation of a modified immune cell expressing a CAR that specifically recognizes mesothelin, IL-7, and CCL19.

FIG. 2 illustrates in vitro killing of MSLN-positive tumor cells by modified immune cells described herein.

FIG. 3 shows in vivo efficacy in a Capan-2 xenograft mouse model using modified immune cells expressing an exemplary 2^{nd} and 3^{rd} generation CAR-T system.

FIG. 4A illustrates histopathological examination of Capan-2 xenografted tumor tissues from mice treated with an exemplary 2^{nd} generation CAR-T system compared to untransduced (UTD) T cells.

FIG. 4B shows in vivo efficacy of an exemplary 2^{nd} generation CAR-T system using a Capan-2 xenograft mouse model.

FIG. 5A shows bioluminescence imaging (BLI) of tumor cell locations in a SKOV3-luc xenograft mouse model in the presence of modified immune cells described herein.

FIG. 5B shows in vivo efficacy of an exemplary 2^{nd} generation CAR-T system using a SKOV3-luc BLI model.

FIG. 6A shows the body weight change of a Capan-2 xenograft mouse model administrated with an exemplary 2nd generation CAR-T system compared to untransduced (UTD) T cells.

FIG. 6B shows the body weight change of non-tumor bearing mice administered with an exemplary 2^{nd} generation CAR-T system compared to untransduced (UTD) T cells.

FIG. 7A shows a histopathological image of lung tissue from a mouse model administered with untransduced (UTD) T cells. The UTD cells had minimal presumptive CAR-T infiltration/inflammation in the lung and spleen.

FIG. 7B shows a histopathological image of lung tissue from a mouse model administered with CAR #364 (2^{nd} 28-28z_7×19). A higher incidence of presumptive CAR-T infiltration/inflammation in the lung, spleen, and liver were observed in the animals compared to the animals administered UTD cells.

FIG. 7C shows a histopathological image of lung tissue from a mouse model administered with CAR #365 (2^{nd} 8-BBz_7×19). A lower incidence of lung mononuclear infiltrates or mixed cell inflammation and a higher incidence of presumptive CAR-T cells engrafting in the spleen and bone marrow were observed in the animals.

FIG. 8A shows flow cytometry analysis of administered T cells in the blood of Capan-2 xenografted mice administered with an exemplary 2^{nd} generation CAR-T system.

FIG. 8B shows flow cytometry analysis of administered T cells in the tumor of Capan-2 xenografted mice administered with an exemplary 2^{nd} generation CAR-T system.

FIG. 8C shows flow cytometry analysis of administered T cells in the spleen of Capan-2 xenografted mice administered with an exemplary 2^{nd} generation CAR-T system.

FIG. 9 shows the human IFN-gamma levels from co-culture supernatants of Capan-2 tumor cells and an exemplary 2^{nd} generation CAR-T system which was pre-incubated with soluble hMSLN.

FIG. 10A shows the components of the 3^{rd} 8-28BBz_7×19 CAR-T (CAR #348) and 2^{nd} 8-BBz_7×19 CAR-T (CAR #365).

FIG. 10B shows in vivo efficacy of the 3^{rd} 8-28BBz_7×19 CAR-T (CAR #348) and 2^{nd} 8-BBz_7×19 CAR-T (CAR #365) using a HepG2-RedFluc xenograft model.

FIG. 10C shows the body weight change of a HepG2-RedFluc xenograft mouse model with 8-28BBz_7×19 CAR-T (CAR #348) and 2^{nd} 8-BBz_7×19 CAR-T (CAR #365).

FIG. 11 shows bioluminescence imaging (BLI) of tumor cell locations in a HepG2-RedFluc xenograft mouse model in the presence of 3^{rd} 8-28BBz_7×19 CAR-T (CAR #348) and 2^{nd} 8-BBz_7×19 CAR-T (CAR #365).

FIG. 12A shows in vivo efficacy of PBS control in HepG2-RedFluc xenograft mouse Group 1 (G1).

FIG. 12B shows in vivo efficacy of equivalent total T cell numbers of control untransduced (UTD) at 3M dose in HepG2-RedFluc xenograft mouse Group 2 (G2).

FIG. 12C shows in vivo efficacy of 2^{nd} 8-BBz_7×19 CAR-T (CAR #365) at 0.3M dose in HepG2-RedFluc xenograft mouse Group 3 (G3).

FIG. 12D shows in vivo efficacy of 2^{nd} 8-BBz_7×19 CAR-T (CAR #365) at 1M dose in HepG2-RedFluc xenograft mouse Group 3 (G4).

FIG. 12E shows in vivo efficacy of 2^{nd} 8-BBz_7×19 CAR-T (CAR #365) at 3M dose in HepG2-RedFluc xenograft mouse Group 3 (G5).

FIG. 12F shows in vivo efficacy of 3^{rd} 8-28BBz_7×19 CAR-T (CAR #348) at 0.3M dose in HepG2-RedFluc xenograft mouse Group 3 (G6).

FIG. 12G shows in vivo efficacy of 3^{rd} 8-28BBz_7×19 CAR-T (CAR #348) at 1M dose in HepG2-RedFluc xenograft mouse Group 3 (G7).

FIG. 12H shows in vivo efficacy of 3^{rd} 8-28BBz_7×19 CAR-T (CAR #348) at 3M dose in HepG2-RedFluc xenograft mouse Group 3 (G8).

### DETAILED DESCRIPTION OF THE DISCLOSURE

### Engineered Immune Cells

In certain embodiments, disclosed herein are engineered immune cells that express an engineered cell surface molecule that specifically binds to mesothelin, interleukin 7 (IL-7), and chemokine (C—C motif) ligand 19 (CCL19). In some embodiments, the engineered cell surface molecule comprises a chimeric antigen receptor (CAR) that specifically recognizes mesothelin or a T cell receptor (TCR) that specifically binds to mesothelin.

In some embodiments, the engineered immune cell contains an exogenous nucleic acid encoding the engineered cell surface molecule, an exogenous nucleic acid encoding IL-7, and an exogenous nucleic acid encoding CCL19. In some embodiments, the engineered immune cell expresses a surface molecule that specifically recognizes mesothelin, IL-7, and CCL19.

Mesothelin (MSLN) is a cell surface-bound glycosylphosphatidylinositol (GPI) anchored protein, in which normal expression is restricted to the mesothelial cells such as from the pleura, pericardium, peritoneum, tunica vaginalis, ovaries, or fallopian tubes. However, MSLN has also been shown to be overexpressed in a plethora of cancers, such as malignant mesothelioma, ovarian cancer, breast cancer (e.g., triple-negative breast cancer, TNBC), pancreatic cancer, lung cancer, gastric cancer, endometrial cancer, cervical cancer, biliary cancer, uterine serous carcinoma, cholangiocarcinoma, and pediatric acute myeloid leukemia. Further, increased MSLN expression has been associated with a poorer prognosis in patients with TNBC, ovarian cancer, lung adenocarcinoma, cholangiocarcinoma, and pancreatic adenocarcinoma.

The physiological and biological functions of MSLN have not been fully elucidated. However, MSLN have been shown to be involved in several mechanism of cancer pathogenesis. For example, in epithelial ovarian carcinoma, patients who exhibited higher levels of MSLN mRNA expression in surgery-resected ovarian cancer tissues showed resistance to chemotherapy with platinum and cyclophosphamide when compared with chemo-sensitive patients who expressed lower MSLN levels (Tang, et al., “The role of mesothelin in tumor progression and targeted therapy,” Anticancer Agents Med Chem. 13(2): 276-280 (2013)). MSLN has also been found to bind with high affinity to the surface mucin MUC16 (or CA125) and the binding has been suggested to mediate adhesion of ovarian cancer cells to the mesothelial cells and promote metastasis (Rump, et al., “Binding of ovarian cancer antigen CA125/MUC16 to mesothelin mediates cell adhesion,” J Biol Chem 279(10): 9190-9198, 2004; Gubbels, et al., “Mesothelin-MUC16 binding is a high affinity, N-glycan dependent interaction that facilitates peritoneal metastasis of ovarian tumors,” Mol Cancer 5(1): 50, 2006). Further, MSLN has been shown to be involved in tumor progression, cell survival and proliferation in pancreatic cancer both in vitro and in vivo (Li, et al., “Mesothelin is a malignant factor and therapeutic vaccine target for pancreatic cancer,” Mol Cancer Ther. 7(2): 286-296, 2008).

### Chimeric Antigen Receptors (CARs)

### A. Anti-Mesothelin Antibodies

In some embodiments, the engineered cell surface molecule comprises a chimeric antigen receptor (CAR) comprising an antibody that specifically recognizes mesothelin. In some embodiments, the antibody specifically recognizes a mammalian mesothelin, e.g., a rodent mesothelin, a non-human primate mesothelin, or a human mesothelin.

Human mesothelin, a 40 kDa protein, is encoded by the MSLN gene. Sequence information on human mesothelin can be appropriately obtained by the search of a publicly known document or a database such as NCBI (www.ncbi.nlm.nih.gov/guide/). Examples of the amino acid sequence information on human mesothelin can include GenBank accession No. NP_037536.2, AAV87530.1, and their isoforms.

In some embodiments, the anti-mesothelin antibody comprises a heavy chain variable region (VH) comprising or consisting of CDRH1 as set forth in SEQ ID NO: 1, CDRH2 as set forth in SEQ ID NO: 2, and CDRH3 as set forth in SEQ ID NO: 3; and a light chain variable region (VL) comprising or consisting of CDRL1 as set forth in SEQ ID NO: 4, CDRL2 as set forth in SEQ ID NO: 5, and CDRL3 as set forth in SEQ ID NO: 6. See Table 1.

TABLE 1

| | | SEQ |
|---|---|---|
| P4* | SEQUENCES | ID NO: |
| HCDR1 | GDSVSSNSAT | 1 |
| HCDR2 | TYYRSKWYN | 2 |
| HCDR3 | ARGMMTYYYGMDV | 3 |
| LCDR1 | SGINVGPYR | 4 |
| LCDR2 | YKSDSDK | 5 |
| LCDR3 | MIWHSSAAV | 6 |
| VH | QVQLQQSGPGLVTPSQTLSLTCAISGDSVSSNSATWNWIRQSPSRG | 7 |
| | LEWLGRTYYRSKWYNDYAVSVKSRMSINPDTSKNQFSLQLNSVT | |
| | PEDTAVYYCARGMMTYYYGMDVWGQGTTVTVSSGILGS | |
| VL | QPVLTQSSSLSASPGASASLTCTLRSGINVGPYRIYWYQQKPGSPP | 8 |
| | QYLLNYKSDSDKQQGSGVPSRFSGSKDASANAGVLLISGLRSEDE | |
| | ADYYCMIWHSSAAVFGGGTQLTVLS | |

*The anti-mesothelin antibody is also referred to herein as P4.

In some embodiments, the anti-mesothelin antibody comprises a heavy chain variable region (VH) comprising a sequence having about 80%, 85%, 90%, 95%, 96%, 97%, 98%, 99%, or 100% sequence identity to SEQ ID NO: 7; and a light chain variable region (VL) comprising a sequence having about 80%, 85%, 90%, 95%, 96%, 97%, 98%, 99%, or 100% sequence identity to SEQ ID NO: 8. In some embodiments, the anti-mesothelin antibody comprises a heavy chain variable region (VH) comprising a sequence having about 80% sequence identity to SEQ ID NO: 7; and a light chain variable region (VL) comprising a sequence having about 80% sequence identity to SEQ ID NO: 8. In some embodiments, the anti-mesothelin antibody comprises a heavy chain variable region (VH) comprising a sequence having about 85% sequence identity to SEQ ID NO: 7; and a light chain variable region (VL) comprising a sequence having about 85% sequence identity to SEQ ID NO: 8. In some embodiments, the anti-mesothelin antibody comprises a heavy chain variable region (VH) comprising a sequence having about 90% sequence identity to SEQ ID NO: 7; and a light chain variable region (VL) comprising a sequence having about 90% sequence identity to SEQ ID NO: 8. In some embodiments, the anti-mesothelin antibody comprises a heavy chain variable region (VH) comprising a sequence having about 95% sequence identity to SEQ ID NO: 7; and a light chain variable region (VL) comprising a sequence having about 95% sequence identity to SEQ ID NO: 8. In some embodiments, the anti-mesothelin antibody comprises a heavy chain variable region (VH) comprising a sequence having about 96% sequence identity to SEQ ID NO: 7; and a light chain variable region (VL) comprising a sequence having about 96% sequence identity to SEQ ID NO: 8. In some embodiments, the anti-mesothelin antibody comprises a heavy chain variable region (VH) comprising a sequence having about 97% sequence identity to SEQ ID NO: 7; and a light chain variable region (VL) comprising a sequence having about 97% sequence identity to SEQ ID NO: 8. In some embodiments, the anti-mesothelin antibody comprises a heavy chain variable region (VH) comprising a sequence having about 98% sequence identity to SEQ ID NO: 7; and a light chain variable region (VL) comprising a sequence having about 98% sequence identity to SEQ ID NO: 8. In some embodiments, the anti-mesothelin antibody comprises a heavy chain variable region (VH) comprising a sequence having about 99% sequence identity to SEQ ID NO: 7; and a light chain variable region (VL) comprising a sequence having about 99% sequence identity to SEQ ID NO: 8. The anti-mesothelin antibody can comprise a heavy chain variable region (VH) comprising SEQ ID NO: 7 and a light chain variable region (VL) comprising SEQ ID NO: 8. The anti-mesothelin antibody can comprise a heavy chain variable region (VH) consisting of SEQ ID NO: 7 and a light chain variable region (VL) consisting of SEQ ID NO: 8.

In some embodiments, one or more residues within the framework region are modified in the anti-mesothelin antibody, generating the 80%, 85%, 90%, 95%, 96%, 97%, 98%, or 99% sequence identity in either the VH or the VL region. The term ‘framework region” refers to the region of the antibody that excludes the complementarity-determining regions (CDRs). In some embodiments, the anti-mesothelin antibody comprises one or more modifications within the framework region and has a sequence comprising 80%, 85%, 90%, 95%, 96%, 97%, 98%, or 99% sequence identity to SEQ ID NO: 7. In some embodiments, the anti-mesothelin antibody comprises one or more modifications within the framework region and has a sequence comprising 85% sequence identity to SEQ ID NO: 7. In some embodiments, the anti-mesothelin antibody comprises one or more modifications within the framework region and has a sequence comprising 90% sequence identity to SEQ ID NO: 7. In some embodiments, the anti-mesothelin antibody comprises one or more modifications within the framework region and has a sequence comprising 95% sequence identity to SEQ ID NO: 7. In some embodiments, the anti-mesothelin antibody comprises one or more modifications within the framework region and has a sequence comprising 96% sequence identity to SEQ ID NO: 7. In some embodiments, the anti-mesothelin antibody comprises one or more modifications within the framework region and has a sequence comprising 97% sequence identity to SEQ ID NO: 7. In some embodiments, the anti-mesothelin antibody comprises one or more modifications within the framework region and has a sequence comprising 98% sequence identity to SEQ ID NO: 7. In some embodiments, the anti-mesothelin antibody comprises one or more modifications within the framework region and has a sequence comprising 99% sequence identity to SEQ ID NO: 7. In some embodiments, the anti-mesothelin antibody comprises one or more modifications within the framework region and has a sequence comprising 80%, 85%, 90%, 95%, 96%, 97%, 98%, or 99% sequence identity to SEQ ID NO: 8. In some embodiments, the anti-mesothelin antibody comprises one or more modifications within the framework region and has a sequence comprising 85% sequence identity to SEQ ID NO: 8. In some embodiments, the anti-mesothelin antibody comprises one or more modifications within the framework region and has a sequence comprising 90% sequence identity to SEQ ID NO: 8. In some embodiments, the anti-mesothelin antibody comprises one or more modifications within the framework region and has a sequence comprising 95% sequence identity to SEQ ID NO: 8. In some embodiments, the anti-mesothelin antibody comprises one or more modifications within the framework region and has a sequence comprising 96% sequence identity to SEQ ID NO: 8. In some embodiments, the anti-mesothelin antibody comprises one or more modifications within the framework region and has a sequence comprising 97% sequence identity to SEQ ID NO: 8. In some embodiments, the anti-mesothelin antibody comprises one or more modifications within the framework region and has a sequence comprising 98% sequence identity to SEQ ID NO: 8. In some embodiments, the anti-mesothelin antibody comprises one or more modifications within the framework region and has a sequence comprising 99% sequence identity to SEQ ID NO: 8.

In some embodiments, the anti-mesothelin antibody comprises a single-chain variable fragment (scFv) format. In some embodiments, the anti-mesothelin scFv antibody comprises a VH comprising or consisting of CDRH1 as set forth in SEQ ID NO: 1, CDRH2 as set forth in SEQ ID NO: 2, and CDRH3 as set forth in SEQ ID NO: 3; and a VL comprising or consisting of CDRL1 as set forth in SEQ ID NO: 4, CDRL2 as set forth in SEQ ID NO: 5, and CDRL3 as set forth in SEQ ID NO: 6. In some embodiments, the anti-mesothelin scFv antibody comprises a VH comprising a sequence having about 80%, 85%, 90%, 95%, 96%, 97%, 98%, 99%, or 100% sequence identity to SEQ ID NO: 7; and a VL comprising a sequence having about 80%, 85%, 90%, 95%, 96%, 97%, 98%, 99%, or 100% sequence identity to SEQ ID NO: 8.

In some embodiments, the VH and the VL of the anti-mesothelin scFv antibody are connected through a peptide linker. The peptide linker can include 3 or more amino acid residues, for example, from about 3 to about 30, from about 3 to about 20, from 3 to about 10, from about 5 to about 30, from about 5 to about 20, or from about 5 to about 10. The peptide linker can include 3, 4, 5, 6, 7, 8, 9, 10, 15, 20, 25, or 30 amino acid residues.

The peptide linker can include a plurality of poly-alanines, poly-glycines, or a mixture of alanine and glycine residues. The peptide linker can include a (Gly₄Ser)n linker (SEQ ID NO: 43), in which n is an integer from 1 to 10, preferably 1, 2, 3, 4, 5, 6, 7, 8, 9, or 10, further preferably 2, 3, 4, or 5. In some embodiments, the peptide linker comprises GGGGSGGGGSGGGGS (SEQ ID NO: 10). In some instances, the peptide linker comprises SGGGSGGGGSGGGGSGGGGSGGGSLQ (SEQ ID NO: 20). In some instances, the peptide linker comprises SGGSGGGGSGGGSGGGGSLQ (SEQ ID NO: 21). In some instances, the peptide linker comprises GSGGGGSGGGGSGGGGS (SEQ ID NO: 22).

In some embodiments, the anti-mesothelin scFv antibody comprises a sequence comprising about 80%, 85%, 90%, 95%, 96%, 97%, 98%, or 99% sequence identity to QVQLQQSGPGLVTPSQTLSLTCAISGDSVSSNSATWNWIRQSPSRGLEWLGRTYYRS KWYNDYAVSVKSRMSINPDTSKNQFSLQLNSVTPEDTAVYYCARGMMTYYYGMD VWGQGTTVTVSSGILGSGGGGSGGGGSGGGGSQPVLTQSSSLSASPGASASLTCTLR SGINVGPYRIYWYQQKPGSPPQYLLNYKSDSDKQQGSGVPSRFSGSKDASANAGVLL ISGLRSEDEADYYCMIWHSSAAVFGGGTQLTVLS (SEQ ID NO: 9). In some embodiments, the anti-mesothelin scFv antibody comprises a sequence comprising about 85% sequence identity to SEQ ID NO: 9. In some embodiments, the anti-mesothelin scFv antibody comprises a sequence comprising about 90% sequence identity to SEQ ID NO: 9. In some embodiments, the anti-mesothelin scFv antibody comprises a sequence comprising about 95% sequence identity to SEQ ID NO: 9. In some embodiments, the anti-mesothelin scFv antibody comprises a sequence comprising about 96% sequence identity to SEQ ID NO: 9. In some embodiments, the anti-mesothelin scFv antibody comprises a sequence comprising about 97% sequence identity to SEQ ID NO: 9. In some embodiments, the anti-mesothelin scFv antibody comprises a sequence comprising about 98% sequence identity to SEQ ID NO: 9. In some embodiments, the anti-mesothelin scFv antibody comprises a sequence comprising about 99% sequence identity to SEQ ID NO: 9. In some embodiments, the anti-mesothelin scFv antibody comprises SEQ ID NO: 9. In some embodiments, the anti-mesothelin scFv antibody consists of SEQ ID NO: 9.

### B. Signaling Peptide

In some embodiments, a chimeric antigen receptor (CAR) disclosed herein comprises a signaling peptide (e.g., as a leader sequence). The signaling peptide can localize the CAR to the surface of the cell. The signaling peptide can include polypeptides of an immune globulin heavy chain, an immunoglobulin light chain, CD8, T cell receptor α and β chains, CD3ζ, CD28, CD3E, CD45, CD4, CD5, CD8, CD9, CD16, CD22, CD33, CD37, CD64, CD80, CD86, CD134, CD137, ICOS, CD154, or a GITR-derived signal peptide (leader sequence).

In some embodiments, the signaling peptide comprises a sequence comprising about 80%, 85%, 90%, 95%, 96%, 97%, 98%, 99%, or 100% sequence identity to MDWTWRILFLVAAATGAHS (SEQ ID NO: 15). In some embodiments, the signaling peptide comprises a sequence comprising about 85% sequence identity to SEQ ID NO: 15. In some embodiments, the signaling peptide comprises a sequence comprising about 90% sequence identity to SEQ ID NO: 15. In some embodiments, the signaling peptide comprises a sequence comprising about 95% sequence identity to SEQ ID NO: 15. In some embodiments, the signaling peptide comprises a sequence comprising about 96% sequence identity to SEQ ID NO: 15. In some embodiments, the signaling peptide comprises a sequence comprising about 97% sequence identity to SEQ ID NO: 15. In some embodiments, the signaling peptide comprises a sequence comprising about 98% sequence identity to SEQ ID NO: 15. In some embodiments, the signaling peptide comprises a sequence comprising about 99% sequence identity to SEQ ID NO: 15. In some embodiments, the signaling peptide comprises SEQ ID NO: 15. In some embodiments, the signaling peptide consists of SEQ ID NO: 15.

### C. Transmembrane Regions

In some embodiments, the anti-mesothelin antibody is linked to one or more transmembrane and intracellular signaling domains. The transmembrane region can be derived from either a natural or synthetic source. Exemplary transmembrane regions can include polypeptides of transmembrane regions derived from CD8, T cell receptor α and β chains, CD3ζ, CD28, CD3E (CD3 epsilon), CD45, CD4, CD5, CD8, CD9, CD16, CD22, CD33, CD37, CD64, CD80, CD86, CD134, CD137, ICOS, CD154, and GITR. In some embodiments, the transmembrane region comprises a CD8 transmembrane region (e.g., human CD8 transmembrane region).

In some embodiments, the transmembrane region comprises a sequence comprising about 80%, 85%, 90%, 95%, 96%, 97%, 98%, 99%, or 100% sequence identity to IYIWAPLAGTCGVLLLSLVITLYC (SEQ ID NO: 12). In some embodiments, the transmembrane region comprises a sequence comprising about 85% sequence identity to SEQ ID NO: 12. In some embodiments, the transmembrane region comprises a sequence comprising about 90% sequence identity to SEQ ID NO: 12. In some embodiments, the transmembrane region comprises a sequence comprising about 95% sequence identity to SEQ ID NO: 12. In some embodiments, the transmembrane region comprises a sequence comprising about 96% sequence identity to SEQ ID NO: 12. In some embodiments, the transmembrane region comprises a sequence comprising about 97% sequence identity to SEQ ID NO: 12. In some embodiments, the transmembrane region comprises a sequence comprising about 98% sequence identity to SEQ ID NO: 12. In some embodiments, the transmembrane region comprises a sequence comprising about 99% sequence identity to SEQ ID NO: 12. In some embodiments, the transmembrane region comprises SEQ ID NO: 12. In some embodiments, the transmembrane region consists of SEQ ID NO: 12.

In some embodiments, the transmembrane region comprises a sequence comprising about 80%, 85%, 90%, 95%, 96%, 97%, 98%, 99%, or 100% sequence identity to IYIWAPLAGTCGVLLLSLVITLYCN (SEQ ID NO: 28). In some embodiments, the transmembrane region comprises a sequence comprising about 85% sequence identity to SEQ ID NO: 28. In some embodiments, the transmembrane region comprises a sequence comprising about 90% sequence identity to SEQ ID NO: 28. In some embodiments, the transmembrane region comprises a sequence comprising about 95% sequence identity to SEQ ID NO: 28. In some embodiments, the transmembrane region comprises a sequence comprising about 96% sequence identity to SEQ ID NO: 28. In some embodiments, the transmembrane region comprises a sequence comprising about 97% sequence identity to SEQ ID NO: 28. In some embodiments, the transmembrane region comprises a sequence comprising about 98% sequence identity to SEQ ID NO: 28. In some embodiments, the transmembrane region comprises a sequence comprising about 99% sequence identity to SEQ ID NO: 28. In some embodiments, the transmembrane region comprises SEQ ID NO: 28. In some embodiments, the transmembrane region consists of SEQ ID NO: 28.

### D. Extracellular Hinge Region

An extracellular hinge region comprising or consisting of an arbitrary oligopeptide or polypeptide may be located between the cell surface molecule recognizing mesothelin and the transmembrane region. Examples of the length of the extracellular hinge region can include 1 to 100 amino acid residues, preferably 10 to 70, 10 to 50, or 10 to 30 amino acid residues. Exemplary extracellular hinge regions can include hinge regions derived from CD8, CD28, and CD4, and an immune globulin hinge region. In some embodiments, the hinge region comprises the hinge region of human CD8.

In some embodiments, the extracellular hinge region is a CD8 hinge region comprising a sequence having about 80%, 85%, 90%, 95%, 96%, 97%, 98%, 99%, or 100% sequence identity to TTTPAPRPPTPAPTIASQPLSLRPEACRPAAGGAVHTRGLDFACD (SEQ ID NO: 11). In some embodiments, the CD8 hinge region comprises a sequence having about 85% sequence identity to SEQ ID NO: 11. In some embodiments, the CD8 hinge region comprises a sequence having about 90% sequence identity to SEQ ID NO: 11. In some embodiments, the CD8 hinge region comprises a sequence having about 95% sequence identity to SEQ ID NO: 11. In some embodiments, the CD8 hinge region comprises a sequence having about 96% sequence identity to SEQ ID NO: 11. In some embodiments, the CD8 hinge region comprises a sequence having about 97% sequence identity to SEQ ID NO: 11. In some embodiments, the CD8 hinge region comprises a sequence having about 98% sequence identity to SEQ ID NO: 11. In some embodiments, the CD8 hinge region comprises a sequence having about 99% sequence identity to SEQ ID NO: 11. In some embodiments, the CD8 hinge region comprises SEQ ID NO: 11. In some embodiments, the CD8 hinge region consists of SEQ ID NO: 11. In some embodiments, the CD8 hinge region comprises a sequence having PTTTPAPRPPTPAPTIASQPLSLRPEACRPAAGGAVHTRGLDFACD (SEQ ID NO: 29). In some embodiments, the CD8 hinge region comprises a sequence having about 85% sequence identity to SEQ ID NO: 29. In some embodiments, the CD8 hinge region comprises a sequence having about 90% sequence identity to SEQ ID NO: 29. In some embodiments, the CD8 hinge region comprises a sequence having about 95% sequence identity to SEQ ID NO: 29. In some embodiments, the CD8 hinge region comprises a sequence having about 96% sequence identity to SEQ ID NO: 29. In some embodiments, the CD8 hinge region comprises a sequence having about 97% sequence identity to SEQ ID NO: 29. In some embodiments, the CD8 hinge region comprises a sequence having about 98% sequence identity to SEQ ID NO: 29. In some embodiments, the CD8 hinge region comprises a sequence having about 99% sequence identity to SEQ ID NO: 29. In some embodiments, the CD8 hinge region comprises SEQ ID NO: 29. In some embodiments, the CD8 hinge region consists of SEQ ID NO: 29.

In some embodiments, the anti-mesothelin scFv antibody is connected to the hinge region through a peptide linker. The peptide linker can include 3 or more amino acid residues, for example, from about 3 to about 30, from about 3 to about 20, from 3 to about 10, from about 5 to about 30, from about 5 to about 20, or from about 5 to about 10. The peptide linker can include 3, 4, 5, 6, 7, 8, 9, 10, 15, 20, 25, or 30 amino acid residues.

The peptide linker can include a plurality of poly-alanines, poly-glycines, or a mixture of alanine and glycine residues or a mixture of either alanines or glycines with one or more additional amino acids. In some embodiments, the peptide linker comprises AlaAlaAla (“AAA”). In some embodiments, the peptide linker is a triple alanine linker or AlaAlaAla (“AAA”). In some embodiments, the peptide linker comprises ArgAlaAlaAla (“RAAA”) (SEQ ID NO: 30). In some embodiments, the peptide linker is ArgAlaAlaAla (“RAAA”) (SEQ ID NO: 30).

In some embodiments, the anti-mesothelin scFv antibody is connected to the hinge region without a linker.

### E. Immune Cell Activating Signaling Region

In some embodiments, the CAR comprises one or more intracellular signaling regions. The intracellular signaling regions can comprise a region capable of transducing signals into the cell when the cell surface molecule recognizes mesothelin. The intracellular signaling region can comprise at least one or more members selected from intracellular regions of polypeptides of CD28, 4-1BB (CD137), GITR, CD27, OX40, HVEM, CD3ζ, or Fc receptor-associated γ chain. In some embodiments, the intracellular signaling region comprises a polypeptide of a CD28 intracellular region, a polypeptide of a 4-1BB intracellular region, a polypeptide of a CD3 intracellular region, or a combination thereof.

In some embodiments, the CAR comprises a 4-1BB intracellular region. In some embodiments, the 4-1BB intracellular region comprises a sequence comprising about 80%, 85%, 90%, 95%, 96%, 97%, 98%, 99%, or 100% sequence identity to KRGRKKLLYIFKQPFMRPVQTTQEEDGCSCRFPEEEEGGCEL (SEQ ID NO: 13). In some embodiments, the 4-1BB intracellular region comprises a sequence comprising about 85% sequence identity to SEQ ID NO: 13. In some embodiments, the 4-1BB intracellular region comprises a sequence comprising about 90% sequence identity to SEQ ID NO: 13. In some embodiments, the 4-1BB intracellular region comprises a sequence comprising about 95% sequence identity to SEQ ID NO: 13. In some embodiments, the 4-1BB intracellular region comprises a sequence comprising about 96% sequence identity to SEQ ID NO: 13. In some embodiments, the 4-1BB intracellular region comprises a sequence comprising about 97% sequence identity to SEQ ID NO: 13. In some embodiments, the 4-1BB intracellular region comprises a sequence comprising about 98% sequence identity to SEQ ID NO: 13. In some embodiments, the 4-1BB intracellular region comprises a sequence comprising about 99% sequence identity to SEQ ID NO: 13. In some embodiments, the 4-1BB intracellular region comprises SEQ ID NO: 13. In some embodiments, the 4-1BB intracellular region consists of SEQ ID NO: 13.

In some embodiments, the CAR further comprises a CD3ζ intracellular region. In some embodiments, the CD3ζ intracellular region comprises a sequence comprising about 80%, 85%, 90%, 95%, 96%, 97%, 98%, 99%, or 100% sequence identity to RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO: 14). In some embodiments, the CD3ζ intracellular region comprises a sequence comprising about 85% sequence identity to SEQ ID NO: 14. In some embodiments, the CD3ζ intracellular region comprises a sequence comprising about 90% sequence identity to SEQ ID NO: 14. In some embodiments, the CD3ζ intracellular region comprises a sequence comprising about 95% sequence identity to SEQ ID NO: 14. In some embodiments, the CD3ζ intracellular region comprises a sequence comprising about 96% sequence identity to SEQ ID NO: 14. In some embodiments, the CD3ζ intracellular region comprises a sequence comprising about 97% sequence identity to SEQ ID NO: 14. In some embodiments, the CD3ζ intracellular region comprises a sequence comprising about 98% sequence identity to SEQ ID NO: 14. In some embodiments, the CD3ζ intracellular region comprises a sequence comprising about 99% sequence identity to SEQ ID NO: 14. In some embodiments, the CD3ζ intracellular region comprises SEQ ID NO: 14. In some embodiments, the CD3ζ intracellular region consists of SEQ ID NO: 14.

### IL-7 and CCL19

Interleukin 7 (IL-7) is involved in the differentiation of multipotent (pluripotent) hematopoietic stem cells into lymphoid progenitor cells and the proliferation of cells in the lymphoid lineage (e.g., B cells, T cells, and NK cells). IL-7 is produced by non-hematopoietic cells such as stromal cells of the bone marrow, the thymus gland, or a lymphoid organ or tissue. In a cancer setting, administration of IL-7 has been shown to transiently disrupt the homeostasis of both CD8⁺ and CD4⁺ T cells and a decrease in the percentage of CD4⁺CD25TFoxp3⁺ T regulatory cells.

In some embodiments, an immune cell described herein expresses IL-7. In some embodiments, IL-7 comprises a sequence comprising about 80%, 85%, 90%, 95%, 96%, 97%, 98%, 99%, or 100% sequence identity to MFHVSFRYIFGLPPLILVLLPVASSDCDIEGKDGKQYESVLMVSIDQLLDSMKEIGSN CLNNEFNFFKRHICDANKEGMFLFRAARKLRQFLKMNSTGDFDLHLLKVSEGTTILL NCTGQVKGRKPAALGEAQPTKSLEENKSLKEQKKLNDLCFLKRLLQEIKTCWNKIL MGTKEH (SEQ ID NO: 18). In some embodiments, IL-7 comprises a sequence comprising about 85% sequence identity to SEQ ID NO: 18. In some embodiments, IL-7 comprises a sequence comprising about 90% sequence identity to SEQ ID NO: 18. In some embodiments, IL-7 comprises a sequence comprising about 95% sequence identity to SEQ ID NO: 18. In some embodiments, IL-7 comprises a sequence comprising about 96% sequence identity to SEQ ID NO: 18. In some embodiments, IL-7 comprises a sequence comprising about 97% sequence identity to SEQ ID NO: 18. In some embodiments, IL-7 comprises a sequence comprising about 98% sequence identity to SEQ ID NO: 18. In some embodiments, IL-7 comprises a sequence comprising about 99% sequence identity to SEQ ID NO: 18. In some embodiments, IL-7 comprises SEQ ID NO: 18. In some embodiments, IL-7 consists of SEQ ID NO: 18.

Chemokine (C—C motif) ligand 19 (CCL19), also known as EBl1 ligand chemokine (ELC) and macrophage inflammatory protein-3-beta (MIP-3-beta), plays a role in lymphocyte recirculation and homing. CCL19 is expressed by dendritic cells or macrophages of lymph nodes and has a function of initiating the migration of T cells, B cells, or mature dendritic cells via its receptor CCR7.

In some embodiments, an immune cell described herein further expresses CCL19. In some embodiments, CCL19 comprises a sequence comprising about 80%, 85%, 90%, 95%, 96%, 97%, 98%, 99%, or 100% sequence identity to MALLLALSLLVLWTSPAPTLSGTNDAEDCCLSVTQKPIPGYIVRNFHYLLIKDGCRVP AVVFTTLRGRQLCAPPDQPWVERIIQRLQRTSAKMKRRSS (SEQ ID NO: 19). In some embodiments, CCL19 comprises a sequence comprising about 85% sequence identity to SEQ ID NO: 19. In some embodiments, CCL19 comprises a sequence comprising about 90% sequence identity to SEQ ID NO: 19. In some embodiments, CCL19 comprises a sequence comprising about 95% sequence identity to SEQ ID NO: 19. In some embodiments, CCL19 comprises a sequence comprising about 96% sequence identity to SEQ ID NO: 19. In some embodiments, CCL19 comprises a sequence comprising about 97% sequence identity to SEQ ID NO: 19. In some embodiments, CCL19 comprises a sequence comprising about 98% sequence identity to SEQ ID NO: 19. In some embodiments, CCL19 comprises a sequence comprising about 99% sequence identity to SEQ ID NO: 19. In some embodiments, CCL19 comprises SEQ ID NO: 19. In some embodiments, CCL19 consists of SEQ ID NO: 19.

### Additional Immune Function Control Factor

The immune cell of the present invention may further express an additional immune function control factor such as IL-15, CCL21, IL-2, IL-4, IL-12, IL-13, IL-17, IL-18, IP-10, interferon-7, MIP-1alpha, GM-CSF, M-CSF, TGF-beta, or TNF-alpha. In some embodiments, the additional immune function control factor comprises IL-15. In some embodiments, the additional immune function control factor comprises IL-2. In some embodiments, the additional immune function control factor comprises interferon-γ. In some embodiments, the additional immune function control factor comprises GM-CSF. In some embodiments, the additional immune function control factor comprises TGF-beta. In some embodiments, the additional immune function control factor comprises TNF-alpha. In some embodiments, the additional immune function control factor is preferably an immune function control factor other than IL-12.

### Arrangement of Each Region

In certain embodiments, disclosed herein is an isolated nucleic acid molecule comprising one or more polynucleotides that encode the engineered cell surface molecule that specifically bind to mesothelin (e.g., a CAR that specifically binds to mesothelin), IL-7, and CCL19. In some embodiments, the isolated nucleic acid molecule comprises a polynucleotide encoding a CAR comprising an antibody that specifically recognizes human mesothelin, a CD8 hinge region, a CD8 transmembrane region, a 4-1BB intracellular region, and a CD3ζ intracellular region; a polynucleotide that encodes IL-7; and a polynucleotide that encodes CCL19. In some embodiments, the polynucleotides that encode the CAR, the IL-7, and the CCL19 are located on two or more different polynucleotides in the nucleic acid molecule. In other embodiments, the isolated nucleic acid molecule comprises the polynucleotides that encode the CAR and IL-7, the polynucleotides that encode the CAR and CCL19, or the polynucleotide that encode the CAR, IL-7, or CCL19.

In some embodiments, the polynucleotide encoding the CAR comprises a signaling peptide upstream of the antibody that specifically recognizes human mesothelin. In some embodiments, the antibody is linked to the CD8 hinge region by a peptide linker (e.g., AlaAlaAla). In some embodiments, the 4-1BB intracellular region is located upstream of the CD3ζ intracellular region in the polynucleotide.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSAA ATTTPAPRPPTPAPTIASQPLSLRPEACRPAAGGAVHTRGLDFACDIYIWAPLAGTCG VLLLSLVITLYCKRGRKKLLYIFKQPFMRPVQTTQEEDGCSCRFPEEEEGGCELRVKF SRSADAPAYQQGQNQLYNELNLGRREEYDVLDKRRGRDPEMGGKPRRKNPQEGLY NELQKDKMAEAYSEIGMKGERRRGKGHDGLYQGLSTATKDTYDALHMQALPPR (SEQ ID NO: 16). The CAR can comprise a sequence having about 85% sequence identity to SEQ ID NO: 16. The CAR can comprise a sequence having about 90% sequence identity to SEQ ID NO: 16. The CAR can comprise a sequence having about 95% sequence identity to SEQ ID NO: 16. The CAR can comprise a sequence having about 96% sequence identity to SEQ ID NO: 16. The CAR can comprise a sequence having about 97% sequence identity to SEQ ID NO: 16. The CAR can comprise a sequence having about 98% sequence identity to SEQ ID NO: 16. The CAR can comprise a sequence having about 99% sequence identity to SEQ ID NO: 16. The CAR can comprise SEQ ID NO: 16. The CAR can consist of SEQ ID NO: 16.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSRAA ATTTPAPRPPTPAPTIASQPLSLRPEACRPAAGGAVHTRGLDFACDIYIWAPLAGTCG VLLLSLVITLYCKRGRKKLLYIFKQPFMRPVQTTQEEDGCSCRFPEEEEGGCELRVKF SRSADAPAYQQGQNQLYNELNLGRREEYDVLDKRRGRDPEMGGKPRRKNPQEGLY NELQKDKMAEAYSEIGMKGERRRGKGHDGLYQGLSTATKDTYDALHMQALPPR (SEQ ID NO: 31). The CAR can comprise a sequence having about 85% sequence identity to SEQ ID NO: 31. The CAR can comprise a sequence having about 90% sequence identity to SEQ ID NO: 31. The CAR can comprise a sequence having about 95% sequence identity to SEQ ID NO: 31. The CAR can comprise a sequence having about 96% sequence identity to SEQ ID NO: 31. The CAR can comprise a sequence having about 97% sequence identity to SEQ ID NO: 31. The CAR can comprise a sequence having about 98% sequence identity to SEQ ID NO: 31. The CAR can comprise a sequence having about 99% sequence identity to SEQ ID NO: 31. The CAR can comprise SEQ ID NO: 31. The CAR can consist of SEQ ID NO: 31.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSTTT PAPRPPTPAPTIASQPLSLRPEACRPAAGGAVHTRGLDFACDIYIWAPLAGTCGVLLL SLVITLYCKRGRKKLLYIFKQPFMRPVQTTQEEDGCSCRFPEEEEGGCELRVKFSRSA DAPAYQQGQNQLYNELNLGRREEYDVLDKRRGRDPEMGGKPRRKNPQEGLYNELQ KDKMAEAYSEIGMKGERRRGKGHDGLYQGLSTATKDTYDALHMQALPPR (SEQ ID NO: 32). The CAR can comprise a sequence having about 85% sequence identity to SEQ ID NO: 32. The CAR can comprise a sequence having about 90% sequence identity to SEQ ID NO: 32. The CAR can comprise a sequence having about 95% sequence identity to SEQ ID NO: 32. The CAR can comprise a sequence having about 96% sequence identity to SEQ ID NO: 32. The CAR can comprise a sequence having about 97% sequence identity to SEQ ID NO: 32. The CAR can comprise a sequence having about 98% sequence identity to SEQ ID NO: 32. The CAR can comprise a sequence having about 99% sequence identity to SEQ ID NO: 32. The CAR can comprise SEQ ID NO: 32. The CAR can consist of SEQ ID NO: 32.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSPTT TPAPRPPTPAPTIASQPLSLRPEACRPAAGGAVHTRGLDFACDIYIWAPLAGTCGVLL LSLVITLYCKRGRKKLLYIFKQPFMRPVQTTQEEDGCSCRFPEEEEGGCELRVKFSRS ADAPAYQQGQNQLYNELNLGRREEYDVLDKRRGRDPEMGGKPRRKNPQEGLYNEL QKDKMAEAYSEIGMKGERRRGKGHDGLYQGLSTATKDTYDALHMQALPPR (SEQ ID NO: 33). The CAR can comprise a sequence having about 85% sequence identity to SEQ ID NO: 33. The CAR can comprise a sequence having about 90% sequence identity to SEQ ID NO: 33. The CAR can comprise a sequence having about 95% sequence identity to SEQ ID NO: 33. The CAR can comprise a sequence having about 96% sequence identity to SEQ ID NO: 33. The CAR can comprise a sequence having about 97% sequence identity to SEQ ID NO: 33. The CAR can comprise a sequence having about 98% sequence identity to SEQ ID NO: 33. The CAR can comprise a sequence having about 99% sequence identity to SEQ ID NO: 33. The CAR can comprise SEQ ID NO: 33. The CAR can consist of SEQ ID NO: 33.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSTTT PAPRPPTPAPTIASQPLSLRPEACRPAAGGAVHTRGLDFACDIYIWAPLAGTCGVLLL SLVITLYCNKRGRKKLLYIFKQPFMRPVQTTQEEDGCSCRFPEEEEGGCELRVKFSRS ADAPAYQQGQNQLYNELNLGRREEYDVLDKRRGRDPEMGGKPRRKNPQEGLYNEL QKDKMAEAYSEIGMKGERRRGKGHDGLYQGLSTATKDTYDALHMQALPPR (SEQ ID NO: 34). The CAR can comprise a sequence having about 85% sequence identity to SEQ ID NO: 34. The CAR can comprise a sequence having about 90% sequence identity to SEQ ID NO: 34. The CAR can comprise a sequence having about 95% sequence identity to SEQ ID NO: 34. The CAR can comprise a sequence having about 96% sequence identity to SEQ ID NO: 34. The CAR can comprise a sequence having about 97% sequence identity to SEQ ID NO: 34. The CAR can comprise a sequence having about 98% sequence identity to SEQ ID NO: 34. The CAR can comprise a sequence having about 99% sequence identity to SEQ ID NO: 34. The CAR can comprise SEQ ID NO: 34. The CAR can consist of SEQ ID NO: 34.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSPTT TPAPRPPTPAPTIASQPLSLRPEACRPAAGGAVHTRGLDFACDIYIWAPLAGTCGVLL LSLVITLYCNKRGRKKLLYIFKQPFMRPVQTTQEEDGCSCRFPEEEEGGCELRVKFSR SADAPAYQQGQNQLYNELNLGRREEYDVLDKRRGRDPEMGGKPRRKNPQEGLYNE LQKDKMAEAYSEIGMKGERRRGKGHDGLYQGLSTATKDTYDALHMQALPPR (SEQ ID NO: 35). The CAR can comprise a sequence having about 85% sequence identity to SEQ ID NO: 35. The CAR can comprise a sequence having about 90% sequence identity to SEQ ID NO: 35. The CAR can comprise a sequence having about 95% sequence identity to SEQ ID NO: 35. The CAR can comprise a sequence having about 96% sequence identity to SEQ ID NO: 35. The CAR can comprise a sequence having about 97% sequence identity to SEQ ID NO: 35. The CAR can comprise a sequence having about 98% sequence identity to SEQ ID NO: 35. The CAR can comprise a sequence having about 99% sequence identity to SEQ ID NO: 35. The CAR can comprise SEQ ID NO: 35. The CAR can consist of SEQ ID NO: 35.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSAA APTTTPAPRPPTPAPTIASQPLSLRPEACRPAAGGAVHTRGLDFACDIYIWAPLAGTC GVLLLSLVITLYCNKRGRKKLLYIFKQPFMRPVQTTQEEDGCSCRFPEEEEGGCELRV KFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO: 36). The CAR can comprise a sequence having about 85% sequence identity to SEQ ID NO: 36. The CAR can comprise a sequence having about 90% sequence identity to SEQ ID NO: 36. The CAR can comprise a sequence having about 95% sequence identity to SEQ ID NO: 36. The CAR can comprise a sequence having about 96% sequence identity to SEQ ID NO: 36. The CAR can comprise a sequence having about 97% sequence identity to SEQ ID NO: 36. The CAR can comprise a sequence having about 98% sequence identity to SEQ ID NO: 36. The CAR can comprise a sequence having about 99% sequence identity to SEQ ID NO: 36. The CAR can comprise SEQ ID NO: 36. The CAR can consist of SEQ ID NO: 36.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSAA ATTTPAPRPPTPAPTIASQPLSLRPEACRPAAGGAVHTRGLDFACDIYIWAPLAGTCG VLLLSLVITLYCNKRGRKKLLYIFKQPFMRPVQTTQEEDGCSCRFPEEEEGGCELRVK FSRSADAPAYQQGQNQLYNELNLGRREEYDVLDKRRGRDPEMGGKPRRKNPQEGL YNELQKDKMAEAYSEIGMKGERRRGKGHDGLYQGLSTATKDTYDALHMQALPPR (SEQ ID NO: 37). The CAR can comprise a sequence having about 85% sequence identity to SEQ ID NO: 37. The CAR can comprise a sequence having about 90% sequence identity to SEQ ID NO: 37. The CAR can comprise a sequence having about 95% sequence identity to SEQ ID NO: 37. The CAR can comprise a sequence having about 96% sequence identity to SEQ ID NO: 37. The CAR can comprise a sequence having about 97% sequence identity to SEQ ID NO: 37. The CAR can comprise a sequence having about 98% sequence identity to SEQ ID NO: 37. The CAR can comprise a sequence having about 99% sequence identity to SEQ ID NO: 37. The CAR can comprise SEQ ID NO: 37. The CAR can consist of SEQ ID NO: 37.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSAA APTTTPAPRPPTPAPTIASQPLSLRPEACRPAAGGAVHTRGLDFACDIYIWAPLAGTC GVLLLSLVITLYCKRGRKKLLYIFKQPFMRPVQTTQEEDGCSCRFPEEEEGGCELRVK FSRSADAPAYQQGQNQLYNELNLGRREEYDVLDKRRGRDPEMGGKPRRKNPQEGL YNELQKDKMAEAYSEIGMKGERRRGKGHDGLYQGLSTATKDTYDALHMQALPPR (SEQ ID NO: 38). The CAR can comprise a sequence having about 85% sequence identity to SEQ ID NO: 38. The CAR can comprise a sequence having about 90% sequence identity to SEQ ID NO: 38. The CAR can comprise a sequence having about 95% sequence identity to SEQ ID NO: 38. The CAR can comprise a sequence having about 96% sequence identity to SEQ ID NO: 38. The CAR can comprise a sequence having about 97% sequence identity to SEQ ID NO: 38. The CAR can comprise a sequence having about 98% sequence identity to SEQ ID NO: 38. The CAR can comprise a sequence having about 99% sequence identity to SEQ ID NO: 38. The CAR can comprise SEQ ID NO: 38. The CAR can consist of SEQ ID NO: 38.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSRAA APTTTPAPRPPTPAPTIASQPLSLRPEACRPAAGGAVHTRGLDFACDIYIWAPLAGTC GVLLLSLVITLYCKRGRKKLLYIFKQPFMRPVQTTQEEDGCSCRFPEEEEGGCELRVK FSRSADAPAYQQGQNQLYNELNLGRREEYDVLDKRRGRDPEMGGKPRRKNPQEGL YNELQKDKMAEAYSEIGMKGERRRGKGHDGLYQGLSTATKDTYDALHMQALPPR (SEQ ID NO: 39). The CAR can comprise a sequence having about 85% sequence identity to SEQ ID NO: 39. The CAR can comprise a sequence having about 90% sequence identity to SEQ ID NO: 39. The CAR can comprise a sequence having about 95% sequence identity to SEQ ID NO: 39. The CAR can comprise a sequence having about 96% sequence identity to SEQ ID NO: 39. The CAR can comprise a sequence having about 97% sequence identity to SEQ ID NO: 39. The CAR can comprise a sequence having about 98% sequence identity to SEQ ID NO: 39. The CAR can comprise a sequence having about 99% sequence identity to SEQ ID NO: 39. The CAR can comprise SEQ ID NO: 39. The CAR can consist of SEQ ID NO: 39.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSRAA ATTTPAPRPPTPAPTIASQPLSLRPEACRPAAGGAVHTRGLDFACDIYIWAPLAGTCG VLLLSLVITLYCNKRGRKKLLYIFKQPFMRPVQTTQEEDGCSCRFPEEEEGGCELRVK FSRSADAPAYQQGQNQLYNELNLGRREEYDVLDKRRGRDPEMGGKPRRKNPQEGL YNELQKDKMAEAYSEIGMKGERRRGKGHDGLYQGLSTATKDTYDALHMQALPPR (SEQ ID NO: 40). The CAR can comprise a sequence having about 85% sequence identity to SEQ ID NO: 40. The CAR can comprise a sequence having about 90% sequence identity to SEQ ID NO: 40. The CAR can comprise a sequence having about 95% sequence identity to SEQ ID NO: 40. The CAR can comprise a sequence having about 96% sequence identity to SEQ ID NO: 40. The CAR can comprise a sequence having about 97% sequence identity to SEQ ID NO: 40. The CAR can comprise a sequence having about 98% sequence identity to SEQ ID NO: 40. The CAR can comprise a sequence having about 99% sequence identity to SEQ ID NO: 40. The CAR can comprise SEQ ID NO: 40. The CAR can consist of SEQ ID NO: 40.

The polynucleotide can encode a CAR comprising a sequence having about 80%, 85%, 90%, 95%, 96%, 97%, 98%, 99%, or 100% sequence identity to MDWTWRILFLVAAATGAHSQVQLQQSGPGLVTPSQTLSLTCAISGDSVSSNSATWN WIRQSPSRGLEWLGRTYYRSKWYNDYAVSVKSRMSINPDTSKNQFSLQLNSVTPED TAVYYCARGMMTYYYGMDVWGQGTTVTVSSGILGSGGGGSGGGGSGGGGSQPVL TQSSSLSASPGASASLTCTLRSGINVGPYRIYWYQQKPGSPPQYLLNYKSDSDKQQGS GVPSRFSGSKDASANAGVLLISGLRSEDEADYYCMIWHSSAAVFGGGTQLTVLSRAA APTTTPAPRPPTPAPTIASQPLSLRPEACRPAAGGAVHTRGLDFACDIYIWAPLAGTC GVLLLSLVITLYCNKRGRKKLLYIFKQPFMRPVQTTQEEDGCSCRFPEEEEGGCELRV KFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO: 41). The CAR can comprise a sequence having about 85% sequence identity to SEQ ID NO: 41. The CAR can comprise a sequence having about 90% sequence identity to SEQ ID NO: 41. The CAR can comprise a sequence having about 95% sequence identity to SEQ ID NO: 41. The CAR can comprise a sequence having about 96% sequence identity to SEQ ID NO: 41. The CAR can comprise a sequence having about 97% sequence identity to SEQ ID NO: 41. The CAR can comprise a sequence having about 98% sequence identity to SEQ ID NO: 41. The CAR can comprise a sequence having about 99% sequence identity to SEQ ID NO: 41. The CAR can comprise SEQ ID NO: 41. The CAR can consist of SEQ ID NO: 41.

The polynucleotide encoding a CAR described herein can comprise a nucleic acid sequence having about 80%, 85%, 90%, 95%, 96%, 97%, 98%, 99%, or 100% sequence identity to ATGGACTGGACCTGGAGGATCCTGTTTCTGGTGGCCGCCGCCACAGGAGCCCAC AGCCAGGTGCAGCTGCAGCAGAGCGGACCTGGCCTGGTGACACCCAGCCAGACC CTGAGCCTGACCTGTGCCATCTCCGGCGATAGCGTGAGCAGCAACAGCGCCACC TGGAACTGGATCAGGCAGAGCCCCAGCAGAGGACTGGAGTGGCTGGGCAGGAC CTACTACAGGAGCAAGTGGTACAACGACTACGCCGTGAGCGTGAAGAGCAGGAT GAGCATCAACCCCGACACCAGCAAGAACCAGTTCTCCCTGCAGCTGAACTCCGT GACCCCCGAGGACACCGCCGTGTACTACTGCGCCAGGGGCATGATGACCTACTA CTACGGCATGGACGTGTGGGGCCAGGGAACCACCGTGACCGTGAGCAGCGGCAT CCTGGGCAGCGGCGGCGGCGGCAGCGGCGGCGGCGGCAGCGGAGGAGGCGGAA GCCAGCCTGTGCTGACCCAGAGCAGCAGCCTGAGCGCTAGCCCTGGAGCTAGCG CCAGCCTGACCTGCACCCTGAGAAGCGGCATCAACGTGGGCCCCTACAGGATCT ACTGGTACCAGCAGAAGCCTGGCAGCCCCCCCCAGTACCTGCTGAACTACAAGA GCGACAGCGACAAGCAGCAGGGCAGCGGCGTGCCTAGCAGATTCAGCGGCAGC AAGGATGCCAGCGCCAACGCCGGAGTGCTGCTGATCAGCGGCCTGAGGAGCGAG GATGAGGCCGACTACTACTGCATGATCTGGCACAGCAGCGCCGCCGTGTTTGGA GGCGGAACCCAGCTGACCGTGCTGAGCGCGGCCGCAACCACCACCCCCGCCCCT AGACCTCCTACACCCGCTCCCACAATCGCCAGCCAGCCTCTGTCTTTAAGACCCG AGGCTTGTAGACCCGCTGCTGGCGGCGCCGTGCATACCAGAGGACTGGACTTCG CTTGTGACATCTACATCTGGGCTCCTTTAGCCGGCACATGTGGAGTGCTGCTGCT GTCTTTAGTGATCACTTTATACTGCAAGAGGGGTCGTAAGAAGCTGCTGTACATC TTCAAGCAGCCCTTCATGAGGCCCGTGCAGACCACCCAAGAAGAGGACGGCTGC AGCTGTCGTTTTCCCGAAGAGGAGGAGGGCGGCTGCGAGCTGAGGGTGAAGTTC AGCAGAAGCGCCGATGCCCCCGCTTACCAGCAAGGTCAGAACCAGCTGTACAAC GAGCTGAATTTAGGTCGTAGGGAGGAGTACGACGTGCTGGACAAGAGGAGGGG CAGAGACCCCGAAATGGGCGGCAAGCCTCGTAGGAAGAACCCCCAAGAAGGTTT ATACAACGAGCTGCAGAAGGACAAGATGGCCGAGGCCTACAGCGAGATCGGCA TGAAGGGCGAGAGGAGGAGAGGCAAGGGCCACGACGGTTTATACCAAGGTCTG AGCACCGCCACCAAGGACACCTACGATGCTTTACACATGCAAGCTTTACCTCCTC GT (SEQ ID NO: 17). The polynucleotide can comprise a nucleic acid sequence having about 85% sequence identity to SEQ ID NO: 17. The polynucleotide can comprise a nucleic acid sequence having about 90% sequence identity to SEQ ID NO: 17. The polynucleotide can comprise a nucleic acid sequence having about 95% sequence identity to SEQ ID NO: 17. The polynucleotide can comprise a nucleic acid sequence having about 96% sequence identity to SEQ ID NO: 17. The polynucleotide can comprise a nucleic acid sequence having about 97% sequence identity to SEQ ID NO: 17. The polynucleotide can comprise a nucleic acid sequence having about 98% sequence identity to SEQ ID NO: 17. The polynucleotide can comprise a nucleic acid sequence having about 99% sequence identity to SEQ ID NO: 17. The polynucleotide can comprise SEQ ID NO: 17. The polynucleotide can consists of SEQ ID NO: 17.

In some embodiments, the polynucleotide encoding the CAR, the polynucleotide encoding IL-7, and the polynucleotide encoding CCL19 are each independently transcribed under a promoter comprising a polynucleotide encoding a self-cleaving 2A peptide (2A peptide) or an internal ribosome entry site (IRES). In some embodiments, the polynucleotide encoding IL-7, and the polynucleotide encoding CCL19 are each independently transcribed under a promoter comprising a polynucleotide encoding the 2A peptide or IRES.

In some embodiments, the polynucleotide encoding the CAR, the polynucleotide encoding IL-7, and the polynucleotide encoding CCL19 are each independently transcribed under a promoter comprising a polynucleotide encoding the 2A peptide. There are four members in the 2A peptide family: P2A, E2A, F2A, and T2A. P2A is derived from porcine teschovirus-1 2A. E2A is derived from equine rhinitis A virus. F2A is derived from foot-and-mouth disease virus 18. T2A is derived from thosea asigna virus 2A. Exemplary sequences for 2A peptide members include:

P2A-
(SEQ ID NO: 42)
ATNFSLLKQAGDVEENPGP;
E2A-
(SEQ ID NO: 44)
QCTNYALLKLAGDVESNPGP;
F2A-
(SEQ ID NO: 45)
VKQTLNFDLLKLAGDVESNPGP;
and
T2A-
(SEQ ID NO: 46)
EGRGSLLTCGDVEENPGP.

In some embodiments, a peptide linker is further added to the terminus of the 2A peptide, e.g., at the N-terminus. In some embodiments, the peptide linker comprises GSG.

In some embodiments, the polynucleotide encoding the CAR, the polynucleotide encoding IL-7, and the polynucleotide encoding CCL19 are each independently transcribed under a promoter comprising a polynucleotide encoding the P2A peptide. The P2A peptide can comprise ATNFSLLKQAGDVEENPGP (SEQ ID NO: 42). In some embodiment, a peptide linker (e.g. GSG) is further added to the N-terminus of the P2A peptide. In some cases, the P2A comprises GSGATNFSLLKQAGDVEENPGP (SEQ ID NO: 23).

In some embodiments, the polynucleotide encoding the CAR, the polynucleotide encoding IL-7, and the polynucleotide encoding CCL19 are arranged in the nucleic acid molecule from the 5′ terminus to the 3′ terminus as:
the polynucleotide encoding the CAR—the polynucleotide encoding IL-7—the polynucleotide encoding CCL19;
the polynucleotide encoding the CAR—the polynucleotide encoding CCL19—the polynucleotide encoding IL-7;
the polynucleotide encoding IL-7—the polynucleotide encoding the CAR—the polynucleotide encoding CCL19;
the polynucleotide encoding CCL19—the polynucleotide encoding the CAR—the polynucleotide encoding IL-7;
the polynucleotide encoding IL-7—the polynucleotide encoding CCL19—the polynucleotide encoding the CAR; or
the polynucleotide encoding CCL19—the polynucleotide encoding IL-7—the polynucleotide encoding the CAR.

In some embodiment, a polynucleotide encoding a first 2A peptide (located between 1^{st} and 2^{nd} polynucleotides) and a polynucleotide encoding a second 2A peptide (located between 2^{nd} and 3^{rd} polynucleotides) are non-identical (codon-optimized) polynucleotide to prevent unexpected recombination. In some embodiment, a polynucleotide encoding the first P2A peptide comprises GGAAGCGGAGCTACTAACTTCAGCCTGCTGAAGCAGGCTGGAGACGTGGAGGAG AACCCTGGACCC (SEQ ID NO: 26) and a polypeptide encoding the second P2A peptide comprises

(SEQ ID NO: 27)
GGCAGCGGCGCCACCAACTTCTCTCTGCTGAAGCAAGCCGGCGATGTGG
AGGAGAATCCCGGCCCC.

In some embodiments, the polynucleotide encoding the CAR, the polynucleotide encoding IL-7, and the polynucleotide encoding CCL19 described herein can comprise a nucleic acid sequence having about 80%, 85%, 90%, 95%, 96%, 97%, 98%, 99%, or 100% sequence identity to

(SEQ ID NO: 25)
ATGGACTGGACCTGGAGGATCCTGTTTCTGGTGGCCGCCGCCACAGGAG
CCCACAGCCAGGTGCAGCTGCAGCAGAGCGGACCTGGCCTGGTGACACC
CAGCCAGACCCTGAGCCTGACCTGTGCCATCTCCGGCGATAGCGTGAGC
AGCAACAGCGCCACCTGGAACTGGATCAGGCAGAGCCCCAGCAGAGGAC
TGGAGTGGCTGGGCAGGACCTACTACAGGAGCAAGTGGTACAACGACTA
CGCCGTGAGCGTGAAGAGCAGGATGAGCATCAACCCCGACACCAGCAAG
AACCAGTTCTCCCTGCAGCTGAACTCCGTGACCCCCGAGGACACCGCCG
TGTACTACTGCGCCAGGGGCATGATGACCTACTACTACGGCATGGACGT
GTGGGGCCAGGGAACCACCGTGACCGTGAGCAGCGGCATCCTGGGCAGC
GGCGGCGGCGGCAGCGGCGGCGGCGGCAGCGGAGGAGGCGGAAGCCAGC
CTGTGCTGACCCAGAGCAGCAGCCTGAGCGCTAGCCCTGGAGCTAGCGC
CAGCCTGACCTGCACCCTGAGAAGCGGCATCAACGTGGGCCCCTACAGG
ATCTACTGGTACCAGCAGAAGCCTGGCAGCCCCCCCCAGTACCTGCTGA
ACTACAAGAGCGACAGCGACAAGCAGCAGGGCAGCGGCGTGCCTAGCAG
ATTCAGCGGCAGCAAGGATGCCAGCGCCAACGCCGGAGTGCTGCTGATC
AGCGGCCTGAGGAGCGAGGATGAGGCCGACTACTACTGCATGATCTGGC
ACAGCAGCGCCGCCGTGTTTGGAGGCGGAACCCAGCTGACCGTGCTGAG
CGCGGCCGCAACCACCACCCCCGCCCCTAGACCTCCTACACCCGCTCCC
ACAATCGCCAGCCAGCCTCTGTCTTTAAGACCCGAGGCTTGTAGACCCG
CTGCTGGCGGCGCCGTGCATACCAGAGGACTGGACTTCGCTTGTGACAT
CTACATCTGGGCTCCTTTAGCCGGCACATGTGGAGTGCTGCTGCTGTCT
TTAGTGATCACTTTATACTGCAAGAGGGGTCGTAAGAAGCTGCTGTACA
TCTTCAAGCAGCCCTTCATGAGGCCCGTGCAGACCACCCAAGAAGAGGA
CGGCTGCAGCTGTCGTTTTCCCGAAGAGGAGGAGGGCGGCTGCGAGCTG
AGGGTGAAGTTCAGCAGAAGCGCCGATGCCCCCGCTTACCAGCAAGGTC
AGAACCAGCTGTACAACGAGCTGAATTTAGGTCGTAGGGAGGAGTACGA
CGTGCTGGACAAGAGGAGGGGCAGAGACCCCGAAATGGGCGGCAAGCCT
CGTAGGAAGAACCCCCAAGAAGGTTTATACAACGAGCTGCAGAAGGACA
AGATGGCCGAGGCCTACAGCGAGATCGGCATGAAGGGCGAGAGGAGGAG
AGGCAAGGGCCACGACGGTTTATACCAAGGTCTGAGCACCGCCACCAAG
GACACCTACGATGCTTTACACATGCAAGCTTTACCTCCTCGTGGAAGCG
GAGCTACTAACTTCAGCCTGCTGAAGCAGGCTGGAGACGTGGAGGAGAA
CCCTGGACCCTGCATGTTCCATGTGAGCTTCAGGTACATCTTCGGACTG
CCTCCTCTCATCCTGGTCCTCCTCCCCGTGGCCAGCTCCGACTGTGACA
TCGAAGGAAAGGATGGCAAGCAGTACGAAAGCGTGCTGATGGTGAGCAT
CGATCAGCTCCTGGATTCCATGAAGGAAATCGGCTCCAACTGCCTCAAC
AATGAGTTCAACTTTTTTAAGAGGCATATCTGCGACGCCAACAAGGAGG
GCATGTTTCTGTTCAGGGCCGCCAGGAAGCTGAGACAGTTCCTCAAGAT
GAATAGCACCGGCGACTTCGACCTCCATCTGCTGAAGGTGTCCGAGGGA
ACCACCATCCTGCTGAACTGCACCGGCCAAGTGAAGGGAAGAAAACCTG
CTGCCCTGGGCGAGGCTCAGCCTACCAAGAGCCTGGAGGAGAACAAAAG
CCTGAAGGAGCAGAAGAAGCTGAACGACCTGTGCTTCCTCAAGAGGCTC
CTGCAGGAGATTAAGACCTGTTGGAACAAGATCCTGATGGGCACAAAGG
AGCACGGCAGCGGCGCCACCAACTTCTCTCTGCTGAAGCAAGCCGGCGA
TGTGGAGGAGAATCCCGGCCCCATGGCTCTGCTGCTCGCCCTGAGCCTG
CTCGTCCTCTGGACCTCCCCTGCTCCTACCCTGAGCGGCACCAATGACG
CTGAAGACTGCTGCCTGTCCGTGACCCAGAAGCCTATCCCCGGATATAT
CGTGAGGAATTTTCATTACCTCCTGATCAAGGACGGCTGTAGAGTGCCC
GCCGTCGTGTTCACAACACTCAGAGGCAGGCAGCTGTGTGCTCCCCCCG
ACCAGCCTTGGGTGGAGAGAATCATTCAGAGACTGCAAAGGACCTCCGC
TAAGATGAAGAGGAGGTCCAGC

### Vectors

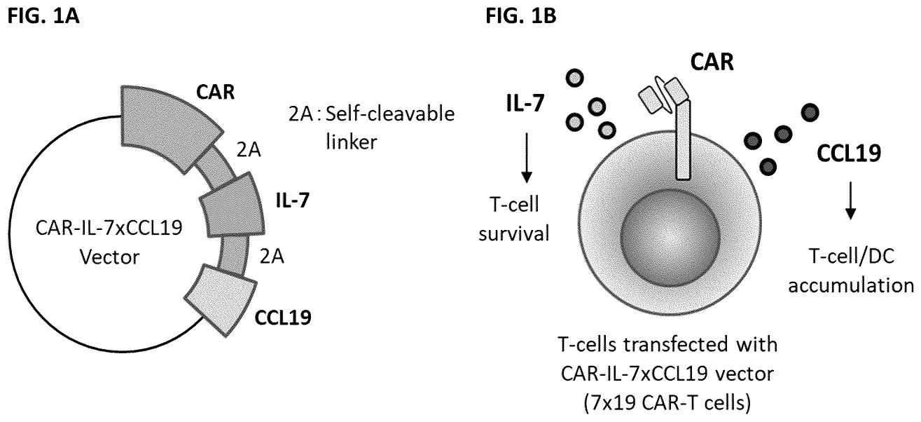

In some embodiments, one or more vectors encompass the polynucleotide encoding the CAR, the polynucleotide encoding IL-7, and the polynucleotide encoding CCL19. In some embodiments, a vector (e.g., an expression vector) comprises the nucleic acid molecule comprising a polynucleotide encoding a chimeric antigen receptor (CAR) comprising an antibody that specifically recognizes human mesothelin, a CD8 hinge region, a CD8 transmembrane region, a 4-1BB intracellular region, and a CD3ζ intracellular region; a polynucleotide encoding IL-7; and a polynucleotide encoding CCL19. See FIG. 1A. In some embodiments, the polynucleotide encoding IL-7, and the polynucleotide encoding CCL19 are arranged in the vector (e.g., an expression vector) from the 5′ terminus to the 3′ terminus as:
the polynucleotide encoding the CAR—the polynucleotide encoding IL-7—the polynucleotide encoding CCL19;
the polynucleotide encoding the CAR—the polynucleotide encoding CCL19—the polynucleotide encoding IL-7;
the polynucleotide encoding IL-7—the polynucleotide encoding the CAR—the polynucleotide encoding CCL19;
the polynucleotide encoding CCL19—the polynucleotide encoding the CAR—the polynucleotide encoding IL-7;
the polynucleotide encoding IL-7—the polynucleotide encoding CCL19—the polynucleotide encoding the CAR; or
the polynucleotide encoding CCL19—the polynucleotide encoding IL-7—the polynucleotide encoding the CAR.

In some embodiments, a first vector (e.g., a first expression vector) comprises the polynucleotide encoding the CAR, and a second vector (e.g., a second expression vector) comprises the polynucleotide encoding IL-7 and the polynucleotide encoding CCL19, in which the polynucleotide encoding IL-7 and the polynucleotide encoding CCL19 are optionally arranged in the second vector (e.g., the second expression vector) from the 5′ terminus to the 3′ terminus as the polynucleotide encoding IL-7—the polynucleotide encoding CCL19 or the polynucleotide encoding CCL19—the polynucleotide encoding IL-7.

In additional embodiments, a first vector (e.g., a first expression vector) comprises the polynucleotide encoding the CAR and either the polynucleotide encoding IL-7 or the polynucleotide encoding CCL19 and a second vector (e.g., a second expression vector) comprises the polynucleotide encoding IL-7 or the polynucleotide encoding CCL19 that is not included in the first vector. In some embodiments, the first vector (e.g., the first expression vector) comprises the polynucleotide encoding the CAR and the polynucleotide encoding IL-7 and the second vector (e.g., the second expression vector) comprises the polynucleotide encoding CCL19. In other embodiments, the first vector (e.g., the first expression vector) comprises the polynucleotide encoding the CAR and the polynucleotide encoding CCL19 and the second vector (e.g., the second expression vector) comprises the polynucleotide encoding IL-7.

In additional embodiments, a first vector (e.g., a first expression vector) comprises the polynucleotide encoding the CAR, a second vector (e.g., a second expression vector) comprises the polynucleotide encoding IL-7, and a third vector (e.g., a third expression vector) comprises the polynucleotide encoding CCL19.

Vectors (e.g., expression vectors) of the present invention may comprise one or more naturally derived nucleic acids or artificially synthesized nucleic acids, and can be appropriately selected according to the type of cells to which the vectors (e.g., the expression vectors) of the present invention are to be introduced. Their sequence information can be appropriately obtained by the search of a publicly known document or a database such as NCBI (www.ncbi.nlm.nih.gov/guide/).

The vector of the present invention can be an expression vector that is introduced into an immune cell or its precursor cell by contacting the vector with the cell so that a predetermined protein (polypeptide) encoded therein can be expressed in the immune cell to produce the modified immune cell of the present invention. The expression vector of the present invention is not particularly limited by any embodiment. Those skilled in the art are capable of designing and producing an expression vector that permits expression of the desired protein (polypeptide) in immune cells. Examples of the expression vector of the present invention comprising a polynucleotide encoding a cell surface molecule specifically recognizing human mesothelin, a polynucleotide encoding IL-7, and a polynucleotide encoding CCL19 can include any of expression vectors for producing the immune cell of the present invention.

The type of expression vector of the present invention may be a linear form or a circular form and may be a non-viral vector such as a plasmid, may be a viral vector, or may be a vector based on a transposon. Such vector may contain a control sequence such as a promoter or a terminator, or a selective marker sequence such as a drug resistance gene or a reporter gene. The polynucleotide encoding the CAR, the polynucleotide encoding IL-7 and the polynucleotide encoding CCL19 can be operably arranged downstream of the promoter sequence so that each of the polynucleotides can be efficiently transcribed.

Examples of the promoter can include: a virus-derived promoter such as retrovirus LTR promoter, SV40 early promoter, cytomegalovirus promoter, and herpes simplex virus thymidine kinase promoter; and a mammal-derived promoter such as phosphoglycerate kinase (PGK) promoter, Xist promoter, 3-actin promoter, and RNA polymerase II promoter. In some embodiment, the promoter can preferably include retrovirus LTR promoter. The retrovirus LTR promoter can comprise CTGAATATGGGCCAAACAGGATATCTGTGGTAAGCAGTTCCTGCCCCGGCTCAG GGCCAAGAACAGATGGAACAGCTGAATATGGGCCAAACAGGATATCTGTGGTAA GCAGTTCCTGCCCCGGCTCAGGGCCAAGAACAGATGGTCCCCAGATGCGGTCCA GCCCTCAGCAGTTTCTAGAGAACCATCAGATGTTTCCAGGGTGCCCCAAGGACCT GAAATGACCCTGTGCCTTATTTGAACTAACCAATCAGTTCGCTTCTCGCTTCTGTT CGCGCGCTTCTGCTCCCCGAGCTCAATAAAAGAGCCCACAACCCCTCACTCGGCG CGCCAGTCCTCCGATTGACTGAGTCGCCCGGGTACCCGTGTATCCAATAAACCCT CTTGCAGTTGCATCCGACTTGTGGTCTCGCTGTTCCTTGGGAGGGTCTCCTCTGAG TGATTGACTACCCGTCAGCGGGGGTCTTTCA (SEQ ID NO: 24). Alternatively, tetracycline-responsive promoter which is induced by tetracycline, Mx1 promoter which is induced by interferon, or the like may be used. Use of the promoter which is induced by a particular substance in the expression vector of the present invention permits control of induction of IL-7 and CCL19 expression according to the course of treatment of cancer, for example, when the immune cell containing the vector of the present invention is used as a pharmaceutical composition for use in the treatment of cancer.

Examples of the viral vector can include a retrovirus vector, a lentivirus vector, an adenovirus vector, and an adeno-associated virus vector and can preferably include a retrovirus vector, e.g., a gamma retrovirus vector, more preferably a pMSGV vector (Tamada k et al., Clin Cancer Res 18: 6436-6445 (2002)), a pMSCV vector (manufactured by Takara Bio Inc.), or a pSFG vector. Use of a retrovirus vector permits long-term and stable expression of a transgene because the transgene is integrated in the genome of a host cell.

One or more assays can be used to confirm the containment of the expression vector of the present invention in the immune cell. Exemplary assays can include flow cytometry for screening the expression of CAR by the engineered immune cells, Northern blotting, Southern blotting, PCR such as RT-PCR, ELISA, or Western blotting. In some embodiments, the expression vector further comprises a marker gene (e.g., encoding a fluorescent protein such as green fluorescent protein (GFP), red fluorescent protein (RFP), or yellow fluorescent protein (YFP)) to detect the expression of the CAR, IL-7, and/or CCL19 by the immune cell.

### Immune Cells and Methods of Production

In certain embodiments, an immune cell described herein is modified to express a cell surface molecule that specifically recognizes mesothelin (e.g., human mesothelin), IL-7, and CCL19 (FIG. 1). Exemplary immune cells can include a lymphoid cell such as a T cell, a natural killer cell (NK cell), and a B cell, an antigen presenting cell such as a monocyte, a macrophage, a dendritic cell, or a granulocyte such as a neutrophil, an eosinophil, a basophil, or a mast cell. The immune cell can include a T cell derived from a mammal such as a human, a dog, a cat, a pig, or a mouse, preferably a T cell derived or separated from a human. The immune cell (e.g., a T cell) can be obtained through culturing, e.g., ex vivo culturing, or harvested directly from the mammal. The immune cell is not limited so long as the cell is involved in immune response and can express the cell surface molecule that specifically recognizes mesothelin (e.g., human mesothelin), expresses IL-7, and expresses CCL19. The immune cell can be an autologous cell harvested from a subject in need thereof for subsequent treatment. The immune cell can also be an allogeneic cell or a syngeneic cell, to a subject in need thereof. The immune cell can also be obtained by culturing stem cells (e.g., induced pluripotent stem cells (iPS cells), embryonic stem cells (ES cells)) or progenitor cells under appropriate conditions for inducing and differentiating such cells into the immune cells.

In certain embodiments, disclosed herein is a population of immune cells modified to express a CAR that specifically recognizes mesothelin, IL-7, and CCL19. In some embodiments, the population of immune cells comprises modified T cells (e.g., either expanded ex vivo or harvested from a mammal) that express a CAR that specifically recognizes mesothelin, IL-7, and CCL19. The population of immune cells can comprise about 20%, 30%, 40%, 50%, 60%, 70%, 80%, 90%, 95%, or higher percentage of the modified T cells. The population of immune cells can comprise about 20%, 30%, 40%, 50%, 60%, 70%, 80%, 90%, or 95% modified T cells. The population of immune cells can comprise a substantially pure population of modified T cells. Exemplary T cells can include an alpha-beta T cell, a gamma-delta T cell, a CD8⁺ T cell, a CD4⁺ T cell, a tumor infiltrating T cell, a memory T cell, a naive T cell, and a natural killer T (NKT) cell.

In some embodiments, the population of immune cells modified to express a CAR that specifically recognizes mesothelin, IL-7, and CCL19 comprises less than about 30%, 25%, 20%, 15%, 10%, 5%, or less contaminant cells. As used herein, the term “contaminant cells” refer to cells that do not express a CAR that specifically recognizes mesothelin, IL-7, and CCL19. The contaminant cells can include T cells that do not express a CAR that specifically recognizes mesothelin, IL-7, and CCL19, and other type of immune cells that do not express a CAR that specifically recognizes mesothelin, IL-7, and CCL19. The contaminant cells can also refer to non-immune cells from a body fluid such as blood or bone marrow fluid, derived from a tissue such as a spleen tissue, the thymus gland, or a lymph node, or derived from a cancer tissue such as a primary tumor tissue, metastatic tumor tissue, or cancerous ascites.

Examples of the method for producing the immune cell of the present invention can include a production method of introducing a polynucleotide encoding a cell surface molecule, a polynucleotide encoding IL-7, and a polynucleotide encoding CCL19 to an immune cell. The production method can include a production method as described in, for example, WO2016/056228, WO2017/159736, WO2013/176915, WO2015/120096, WO2016/019300 or Vormittag P et al, Curr Opin Biotechnol 2018; 53: 164-81. Alternative examples can include a method of purifying and obtaining an immune cell from a transgenic mammal produced by implanting a vector for expression of a cell surface molecule specifically recognizing mesothelin (e.g., human mesothelin), IL-7, and/or CCL19 into a fertilized egg, and a production method of further introducing, if necessary, the vector for expression of a cell surface molecule specifically recognizing mesothelin (e.g., human mesothelin), IL-7, and/or CCL19 to the immune cell purified and obtained from the transgenic mammal.

In the case of introducing a polynucleotide encoding a cell surface molecule, a polynucleotide encoding IL-7, and a polynucleotide encoding CCL19, or the vectors described supra, the method can be any method for introducing the polynucleotides or the vectors to the immune cell. Examples can include an electroporation method (Cytotechnology, 3, 133 (1990)), a calcium phosphate method (Japanese unexamined Patent Application Publication No. 2-227075), a lipofection method (Proc. Natl. Acad. Sci. U.S.A., 84, 7413 (1987)), and a viral infection method. Exemplary viral infection methods can include a method of transfecting a packaging cell such as a GP2-293 cell (manufactured by Takara Bio Inc.), a Plat-GP cell (manufactured by Cosmo Bio Co., Ltd.), a PG13 cell (ATCC CRL-10686), or a PA317 cell (ATCC CRL-9078) with the vector to be introduced and a packaging plasmid to produce a recombinant virus, and infecting the immune cell with the recombinant virus (see e.g., WO2017/159736).

In some embodiments, the method comprises introducing one or more vectors comprising the polynucleotide encoding the CAR, the polynucleotide encoding IL-7, and the polynucleotide encoding CCL19 to an immune cell. In some embodiments, the method comprises introducing a vector (e.g., an expression vector) comprising the nucleic acid molecule comprising a polynucleotide encoding a chimeric antigen receptor (CAR) comprising an antibody that specifically recognizes human mesothelin, a CD8 hinge region, a CD8 transmembrane region, a 4-1BB intracellular region, and a CD3ζ intracellular region; a polynucleotide encoding IL-7; and a polynucleotide encoding CCL19 to an immune cell. In some embodiments, the method comprises introducing a first vector (e.g., a first expression vector) comprising the polynucleotide encoding the CAR and a second vector (e.g., a second expression vector) comprising the polynucleotide encoding IL-7 and the polynucleotide encoding CCL19, either together or in stages, to an immune cell. In some embodiments, the method comprises introducing a first vector (e.g., a first expression vector) comprising the polynucleotide encoding the CAR and either the polynucleotide encoding IL-7 or the polynucleotide encoding CCL19 and a second vector (e.g., a second expression vector) comprising the polynucleotide encoding IL-7 or the polynucleotide encoding CCL19 that is not included in the first vector, either together or in stages, to an immune cell. In some embodiments, the method comprises introducing a first vector (e.g., a first expression vector) comprising the polynucleotide encoding the CAR and the polynucleotide encoding IL-7 and a second vector (e.g., a second expression vector) comprising the polynucleotide encoding CCL19, either together or in stages, to an immune cell. In some embodiments, the method comprises introducing a first vector (e.g., a first expression vector) comprising the polynucleotide encoding the CAR and the polynucleotide encoding CCL19 and a second vector (e.g., a second expression vector) comprising the polynucleotide encoding IL-7, either together or in stages, to an immune cell. In some embodiments, the method comprises introducing a first vector (e.g., a first expression vector) comprising the polynucleotide encoding the CAR, a second vector (e.g., a second expression vector) comprising the polynucleotide encoding IL-7, and a third vector (e.g., a third expression vector) comprising the polynucleotide encoding CCL19, either together or in stages, to an immune cell.

One or more of the polynucleotide encoding the CAR, the polynucleotide encoding IL-7, and the polynucleotide encoding CCL19 can be integrated into the genome of the immune cell. In some embodiments, the polynucleotide encoding the CAR, the polynucleotide encoding IL-7, and the polynucleotide encoding CCL19 are not integrated into the genome (e.g., episomally).

### Methods of Use

In certain embodiments, disclosed herein is a method of treating a mesothelin-expressing cancer. In some embodiments, the method comprises administering to a subject in need thereof an immune cell described herein modified to express an engineered cell surface molecule that specifically binds to mesothelin, interleukin 7 (IL-7), and chemokine (C—C motif) ligand 19 (CCL19). In some embodiments, the immune cell is modified to express an engineered cell surface molecule comprises a chimeric antigen receptor (CAR) that specifically recognizes mesothelin or a T cell receptor (TCR) that specifically binds to mesothelin. In some embodiments, the immune cell is modified to express a CAR comprising an antibody that specifically recognizes human mesothelin, a CD8 hinge region, a CD8 transmembrane region, a 4-1BB intracellular region and a CD3ζ intracellular region; IL-7; and CCL19.

In some embodiments, the mesothelin-expressing cancer is a solid tumor. In some embodiments, the solid tumor comprises mesothelioma, colorectal cancer, pancreatic cancer, thymic cancer, bile duct cancer, lung cancer, skin cancer, breast cancer, prostate cancer, urinary bladder cancer, virginal cancer, neck cancer, uterine cancer, liver cancer, kidney cancer, gastric cancer, spleen cancer, tracheal cancer, bronchial cancer, stomach cancer, esophageal cancer, gallbladder cancer, testis cancer, ovarian cancer, or bone cancer. In some embodiments, the mesothelin-expressing cancer is ovarian cancer. In some embodiments, the mesothelin-expressing cancer is mesothelioma. In some embodiments, the mesothelin-expressing cancer is gastric cancer. In some embodiments, the mesothelin-expressing cancer is lung cancer (e.g., non-small cell lung cancer (NSCLC), small cell lung cancer (SCLC), lung carcinoid tumors, adenosquamoous carcinoma of the lung, large cell neuroendocrine carcinoma, or salivary gland-type lung carcinoma). In some embodiments, the mesothelin-expressing cancer is NSCLC (e.g., adenocarcinoma of the lung, squamous cell, large-cell undifferentiated carcinoma, sarcomatoid carcinoma, or adenosquamous carcinoma).

The mesothelin-expressing cancer can be a hematopoietic cancer. The hematopoietic cancer can be a B-cell hematopoietic cancer, a T-cell hematopoietic cancer, a Hodgkin's lymphoma, or a non-Hodgkin's lymphoma. The hematopoietic cancer can be acute lymphocytic leukemia (ALL), chronic lymphocytic leukemia (CLL), small lymphocytic lymphoma (SLL), diffuse large B-cell lymphoma (DLBCL), follicular lymphoma (FL), mantle cell lymphoma (MCL), marginal zone lymphomas, Burkitt lymphoma, or Waldenstrom macroglobulinemia.

The hematopoietic cancer can be a sarcoma. The sarcoma can include chondrosarcoma, Ewing's sarcoma, malignant hemangioendothelioma, malignant schwannoma, osteosarcoma, or soft tissue sarcoma.

The mesothelin-expressing cancer can be a metastatic cancer, e.g., a metastatic solid tumor or a metastatic hematopoietic cancer. The metastatic mesothelin-expressing cancer can be a metastatic ovarian cancer, metastatic mesothelioma, metastatic gastric cancer, or a metastatic lung cancer (e.g., metastatic NSCLC).

The mesothelin-expressing cancer can be a relapsed or refractory cancer, e.g., a relapsed or refractory solid tumor, or a relapsed or refractory hematopoietic cancer. The relapsed or refractory mesothelin-expressing cancer can be a relapsed or refractory ovarian cancer, relapsed or refractory mesothelioma, relapsed or refractory gastric cancer, or a relapsed or refractory lung cancer (e.g., relapsed or refractory NSCLC).

In some embodiments, the method further comprises administering to the subject an additional therapeutic agent or an additional therapeutic regimen. The additional therapeutic agent can comprise a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some embodiments, the additional therapeutic agent comprises a first-line therapy. As used herein, “first-line therapy” comprises a primary treatment for a subject with a cancer. In some embodiments, the cancer is a primary cancer. In other embodiments, the cancer is a metastatic or recurrent cancer. In some embodiments, the first-line therapy comprises chemotherapy. In other embodiments, the first-line treatment comprises radiation therapy. A skilled artisan would readily understand that different first-line treatments may be applicable to different type of cancers.

In some embodiments, the additional therapeutic agent comprises an inhibitor of the enzyme poly ADP ribose polymerase (PARP). Exemplary PARP inhibitors include, but are not limited to, olaparib (AZD-2281, Lynparza®, from Astra Zeneca), rucaparib (PF-01367338, Rubraca®, from Clovis Oncology), niraparib (MK-4827, Zejula®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some embodiments, the additional therapeutic agent comprises an immune checkpoint inhibitor. In some embodiments, the checkpoint inhibitor comprises pembrolizumab, nivolumab, tremelimumab, or ipilimumab. In some embodiments, the checkpoint inhibitor comprises an inhibitor of PD-L1, PD-L2, PD-1, CTLA-4, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. The inhibitor can be an antibody or fragments (e.g., a monoclonal antibody, a human, humanized, or chimeric antibody) thereof, a RNAi molecule, or a small molecule.

In some embodiments, the additional therapeutic agent comprises an antibody such as alemtuzumab, trastuzumab, ibritumomab tiuxetan, brentuximab vedotin, ado-trastuzumab emtansine, or blinatumomab.

In some embodiments, the additional therapeutic agent comprises a cytokine. Exemplary cytokines include, but are not limited to, IL-Iβ, IL-6, IL-7, IL-10, IL-12, IL-15, IL-21, or TNFα.

In some embodiments, the additional therapeutic agent comprises a receptor agonist. In some embodiments, the receptor agonist comprises a Toll-like receptor (TLR) ligand. In some embodiments, the TLR ligand comprises TLR1, TLR2, TLR3, TLR4, TLR5, TLR6, TLR7, TLR8, or TLR9. In some embodiments, the TLR ligand comprises a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some embodiments, the additional therapeutic agent comprises fludarabine and cyclophosphamide.

In some embodiments, the additional therapeutic agent comprises tisagenlecleucel (KYMRIAH®), axicabtagene ciloleucel (YESCARTA®), or brexucabtagene autoleucel (TECARTUS®).

In some embodiments, the additional therapeutic regimen comprises surgery.

In some embodiments, the immune cell described herein or the pharmaceutical composition described herein and the additional therapeutic agent are administered simultaneously.

In some embodiments, the immune cell described herein or the pharmaceutical composition described herein and the additional therapeutic agent are administered sequentially. In some embodiments, the immune cell described herein or the pharmaceutical composition described herein is administered to the subject prior to administration of the additional therapeutic agent. In other embodiments, the immune cell described herein or the pharmaceutical composition described herein is administered to the subject after administration of the additional therapeutic agent.

In some embodiments, the subject is a human.

In some embodiments, also described herein is a method for producing an immune cell expressing cell surface molecules that specifically recognizes mesothelin (e.g., human mesothelin), IL-7, and CCL19. The method comprises introducing a nucleic acid molecule described herein or the vector comprising the nucleic acid molecule to an immune cell to induce expression of cell surface molecules that specifically recognize human mesothelin, IL-7, and CCL19 by the immune cell. In some embodiments, the immune cell is a T cell, a natural killer (NK) cell, a B cell, an antigen presenting cell, or a granulocyte, optionally a T cell or an NK cell.

### Pharmaceutical Composition

In certain embodiments, the immune cells described above are formulated as a pharmaceutical composition. In some embodiments, the pharmaceutical composition is administered to a subject by multiple administration routes, including but not limited to, parenteral, oral, sublingual, or transdermal administration routes. In some embodiments, parenteral administration comprises intravenous, subcutaneous, intramuscular, intranasal, intra-arterial, intra-articular, intradermal, intraosseous infusion, intraperitoneal, subarachnoidal, intracranial, intrasynovial, intratumoral, intracutaneous, intramedullary, intracardiac, or intratechal administration. In some embodiments, the pharmaceutical composition is formulated for local administration. In other embodiments, the pharmaceutical composition is formulated for systemic administration.

In some embodiments, the pharmaceutical composition comprises a pharmaceutically acceptable additive. Examples of the additive can include saline, buffered saline, a cell culture medium, dextrose, injectable water, glycerol, ethanol, a stabilizer, a solubilizer, a surfactant, a buffer, an antiseptic, a tonicity agent, a filler, a lubricant, or a combination thereof.

In some embodiments, the pharmaceutical composition further comprises pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some embodiments, the pharmaceutical composition includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In an exemplary method, the pharmaceutical composition of the present invention can be independently administered in one portion or several divided portions 4 times, 3 times, twice, or once a day, at a 1-day, 2-day, 3-day, 4-day, or 5-day interval, once a week, at a 7-day, 8-day, or 9-day interval, twice a week, once a month, twice a month, three times per month, or more.

In the case wherein the patient's status does improve, upon the doctor's discretion the administration of the composition is given continuously, alternatively, the dose of the composition being administered is temporarily reduced or temporarily suspended for a certain length of time (i.e., a “drug holiday”). In some embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In some embodiments, the amount of a given modified T-cells that correspond to such an amount varies depending upon factors such as the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some embodiments, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Articles of Manufacture

In certain embodiments, disclosed herein is a kit comprising a nucleic acid molecule described above, a vector comprising the nucleic acid molecule described above, an immune cell expressing a CAR that specifically recognizes mesothelin (e.g., human mesothelin), IL-7, and CCL19, or a pharmaceutical composition. In some embodiments, the kit may contain one or more packing materials such as a package insert, a label, a package, or the like stating a use method, etc. for use in the treatment of cancer. Since the immune cell in the pharmaceutical composition of the present invention has suppressive effects on tumor recurrence, the pharmaceutical composition of the present invention may serve as a pharmaceutical composition for use in the suppression of tumor recurrence. Such a pharmaceutical composition for use in the suppression of tumor recurrence may contain one or more packing materials such as a package insert, a label, a package, or the like stating a use method, etc. for use in the suppression of tumor recurrence.

The term “packing material” refers to a physical structure housing a component of the kit. The material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., floppy diskette, ZIP disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein (e.g., the binding agent or pharmaceutical composition), dose amounts, clinical pharmacology of the active agent(s) including mechanism of action, pharmacokinetics and pharmacodynamics. Labels or inserts can include information identifying manufacturer information, lot numbers, and location and date of manufacture.

Labels or inserts can include information on a disease for which a kit component may be used. Labels or inserts can include instructions for the clinician or subject for using one or more of the kit components in a method, or treatment protocol or therapeutic regimen. Instructions can include dosage amounts, frequency or duration, and instructions for practicing any of the methods, treatment protocols or therapeutic regimes described herein.

Labels or inserts can include information on any benefit that a component may provide, such as a therapeutic benefit. Labels or inserts can include information on potential adverse side effects, such as warnings to the subject or clinician regarding situations where it would not be appropriate to use a particular composition (e.g., a modified immune cell described herein). For example, adverse side effects are generally more likely to occur at higher dose amounts, frequency or duration of the active agent and, therefore, instructions could include recommendations against higher dose amounts, frequency or duration. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

Also as used herein, “and/or” refers to and encompasses any and all possible combinations of one or more of the associated listed items, as well as the lack of combinations when interpreted in the alternative (“or”).

As used herein, “optional” or “optionally” means that the subsequently described event or circumstance can or cannot occur, and that the description includes embodiments where the event or circumstance occurs and embodiments where it does not.

As used herein, the term “antibody” refers to a protein that binds to other molecules (antigens, e.g., mesothelin) via heavy and light chain variable domains, VH and VL, respectively. The term “variable region” or “variable domain” refers to the domain of an antibody that is involved in binding the antibody to antigen. The variable domains of the heavy chain and light chain (VH and VL, respectively) of a native antibody generally have similar structures, with each domain comprising four conserved framework regions (FRs) and three CDRs. (See, e.g., Kindt et al. Kuby Immunoloy, 6th ed., W.H. Freeman and Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Antibodies of the disclosure include monoclonal antibodies. The term “monoclonal,” when used in reference to an antibody refers to an antibody that is based upon, obtained from or derived from a single clone, including any eukaryotic, prokaryotic, or phage clone. A “monoclonal” antibody is therefore defined herein structurally, and not the method by which it is produced.

Monoclonal antibodies are made by methods known in the art (Kohler et al., Nature, 256:495(1975); and Harlow and Lane, Using Antibodies: A Laboratory Manual, Cold Spring Harbor Laboratory, 1999). Briefly, monoclonal antibodies can be obtained by injecting mice with antigen. The polypeptide or peptide used to immunize an animal may be derived from translated DNA or chemically synthesized and conjugated to a carrier protein. Commonly used carriers which are chemically coupled to the immunizing peptide include, for example, keyhole limpet hemocyanin (KLH), thyroglobulin, bovine serum albumin (BSA), and tetanus toxoid. Antibody production is verified by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from hybridoma cultures. Monoclonal antibodies can be isolated and purified from hybridoma cultures by a variety of established techniques which include, for example, affinity chromatography with Protein-A Sepharose, size-exclusion chromatography, and ion-exchange chromatography (see e.g., Coligan et al., Current Protocols in Immunology sections 2.7.1-2.7.12 and sections 2.9.1-2.9.3; and Barnes et al., “Methods in Molecular Biology,” 10:79-104, Humana Press (1992)).

Antibodies of the disclosure can belong to any antibody class, IgM, IgG, IgE, IgA, IgD, or subclass. Exemplary subclasses for IgG are IgG₁, IgG₂, IgG₃ and IgG₄.

Antibodies of the disclosure can be a humanized antibody. The term “humanized” refers to an antibody sequence that has non-human amino acid residues of one or more complementarity determining regions (CDRs) that specifically bind to the antigen in an acceptor human immunoglobulin molecule, and one or more human amino acid residues in the framework region (FR) that flank the CDRs. Any mouse, rat, guinea pig, goat, non-human primate (e.g., ape, chimpanzee, macaque, orangutan, etc.) or other animal antibody may be used as a CDR donor for producing humanized antibody. Human framework region residues can be replaced with corresponding non-human residues (e.g., from the donor variable region). Residues in the human framework regions can therefore be substituted with a corresponding residue from the non-human CDR donor antibody. A humanized antibody may include residues, which are found neither in the human antibody nor in the donor CDR or framework sequences. The use of antibody components derived from humanized monoclonal antibodies reduces problems associated with the immunogenicity of non-human regions. Methods of producing humanized antibodies are known in the art (see, for example, U.S. Pat. Nos. 5,225,539; 5,530,101, 5,565,332 and 5,585,089; Riechmann et al., (1988) Nature 332:323; EP 239,400; WO91/09967; EP 592,106; EP 519,596; Padlan Molecular Immunol. (1991) 28:489; Studnicka et al., Protein Engineering (1994) 7:805; Singer et al., J. Immunol. (1993) 150:2844; and Roguska et al., Proc. Nat'l. Acad. Sci. USA (1994) 91:969).

Antibodies of the disclosure can be a chimeric antibody. The term “chimeric antibody” refers to an antibody in which different portions are derived from different animal species, such as those having a variable region derived from a murine monoclonal antibody and a human immunoglobulin constant region, e.g., humanized antibodies. In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used.

Antibodies of the disclosure include binding fragments thereof. Exemplary antibody fragments include Fab, Fab′, F(ab′)₂, Fv, Fd, single-chain Fv (scFv), disulfide-linked Fvs (sdFv), light chain variable region V_{L}, heavy chain variable region V_{H}, trispecific (Fab₃), bispecific (Fab₂), diabody ((V_{L}-V_{H})₂ or (V_{H}-V_{L})₂), triabody (trivalent), tetrabody (tetravalent), minibody ((scF_{V}-C_{H})₂), bispecific single-chain Fv (Bis-scFv), IgGdeltaCH2, scFv-Fc, (scFv)₂-Fc and IgG4PE. Such fragments can have the binding affinity as the full length antibody, the binding specificity as the full length antibody, or one or more activities or functions of as a full length antibody, e.g., a function or activity of mesothelin binding antibody.

Antibody fragments can be combined. For example, a V_{L} or V_{H} subsequences can be joined by a linker sequence thereby forming a V_{L}-V_{H} chimera. A combination of single-chain Fvs (scFv) sequences can be joined by a linker sequence thereby forming a scFv-scFv chimera. Antibody fragments include single-chain antibodies or variable region(s) alone or in combination with all or a portion of other sequences.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

Antibody fragments can also be prepared by proteolytic hydrolysis of the antibody, for example, by pepsin or papain digestion of whole antibodies. Antibody fragments produced by enzymatic cleavage with pepsin provide a 5S fragment denoted F(ab′)₂. This fragment can be further cleaved using a thiol reducing agent to produce 3.5S Fab′ monovalent fragments. Alternatively, an enzymatic cleavage using pepsin produces two monovalent Fab′ fragments and the Fc fragment directly (see, e.g., U.S. Pat. Nos. 4,036,945 and 4,331,647; and Edelman et al., Methods Enzymol. 1:422 (1967)). Other methods of cleaving antibodies, such as separation of heavy chains to form monovalent light-heavy chain fragments, further cleavage of fragments, or other enzymatic or chemical may also be used.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

As used herein, “identical”, “sequence identity”, or percent “identity”, when used in the context of two or more nucleic acids or polypeptide sequences, refers to two or more sequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region. The alignment and sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “identical”, “sequence identity”, or percent “identity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The term “protein”, “peptide”, and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. Polypeptides include full length native polypeptide, and “modified” forms such as subsequences, variant sequences, fusion/chimeric sequences and dominant-negative sequences. As used herein the term “amino acid” refers to either natural and/or unnatural or synthetic amino acids, including glycine and both the D and L optical isomers, amino acid analogs and peptidomimetics.

Peptides include L- and D-isomers, and combinations thereof. Peptides can include modifications typically associated with post-translational processing of proteins, for example, cyclization (e.g., disulfide or amide bond), phosphorylation, glycosylation, carboxylation, ubiquitination, myristylation, or lipidation. Modified peptides can have one or more amino acid residues substituted with another residue, added to the sequence or deleted from the sequence. Specific examples include one or more amino acid substitutions, additions or deletions (e.g., 1-3, 3-5, 5-10, 10-20, or more).

As used herein, the terms “modification” and “modified” refer to a mutation, substitution, addition, or deletion of one or more amino acid residues of an antibody, protein, or polypeptide in comparison to a reference antibody, protein, or polypeptide that is the equivalent of the antibody, protein, or polypeptide without the modification. In some embodiments, the modification comprises a conservative substitution.

A “conservative substitution” is the replacement of one amino acid by a biologically, chemically or structurally similar residue. Biologically similar means that the substitution is compatible with an activity or function of the unsubstituted sequence. Structurally similar means that the amino acids have side chains with similar length, such as alanine, glycine and serine, or having similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

As used herein, the term “nucleic acid” refers to a DNA or an RNA, comprising natural, synthetic, or artificial nucleotide analogues or bases. In some embodiments, a nucleotide analogue or artificial nucleotide base comprises a nucleic acid with a modification at a 2′ hydroxyl group of the ribose moiety. In some embodiments, the modification includes an H, OR, R, halo, SH, SR, NH₂, NHR, NR₂, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some embodiments, the alkyl moiety further comprises a modification. In some embodiments, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some embodiments, the alkyl moiety further comprises a hetero substitution. In some embodiments, the carbon of the heterocyclic group is substituted by a nitrogen, oxygen or sulfur. In some embodiments, the heterocyclic substitution includes but is not limited to, morpholino, imidazole, and pyrrolidino.

In some embodiments, a nucleotide analogue comprises a modified base such as, but not limited to, 5-propynyluridine, 5-propynylcytidine, 6-methyladenine, 6-methylguanine, N, N, -dimethyladenine, 2-propyladenine, 2propylguanine, 2-aminoadenine, 1-methyl inosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some embodiments are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

The nucleic acid molecules of the present invention can be produced by a publicly known technique such as a chemical synthesis method or a PCR amplification method on the basis of information on the nucleotide sequence of each of the nucleic acids. Codons selected for encoding amino acids may be engineered in order to optimize nucleic acid expression in host cells of interest.

As used herein, the term “substantially” when describing the population of T cells refers to a population comprising less than about 30%, 25%, 20%, 15%, 10%, 5%, or less contaminant cells. In some embodiments, the contaminant cells are less than about 20% in the population of T cells. In some embodiments, the contaminant cells are less than about 15% in the population of T cells. In some embodiments, the contaminant cells are less than about 10% in the population of T cells.

As used herein, the terms “treating,” “treatment” and the like mean obtaining a desired pharmacologic and/or physiologic effect. The effect may be therapeutic in terms of amelioration of the symptoms of the disease, or a partial or complete cure for a disease and/or adverse effect attributable to the disease. In one aspect, the term “treatment” excludes prophylaxis.

As used herein, to “treat” further includes systemic amelioration of the symptoms associated with the pathology and/or a delay in onset of symptoms. Clinical and subclinical evidence of “treatment” will vary with the pathology, the individual and the treatment. In one aspect, treatment excludes prophylaxis.

The term “ameliorate” means a detectable improvement in a subject's condition. A detectable improvement includes a subjective or objective decrease, reduction, inhibition, suppression, limit or control in the occurrence, frequency, severity, progression, or duration of a symptom caused by or associated with a disease, such as one or more adverse symptoms, disorders, illnesses, pathologies, diseases, or complications caused by or associated with the disease, or an improvement in an underlying cause or a consequence of the disease, or a reversal of the disease.

Treatment can therefore result in decreasing, reducing, inhibiting, suppressing, limiting, controlling or preventing a disease, or an associated symptom or consequence, or underlying cause; decreasing, reducing, inhibiting, suppressing, limiting, controlling or preventing a progression or worsening of a disease, condition, symptom or consequence, or underlying cause; or further deterioration or occurrence of one or more additional symptoms of the disease condition, or symptom. Thus, a successful treatment outcome leads to a “therapeutic effect,” or “benefit” of decreasing, reducing, inhibiting, suppressing, limiting, controlling or preventing the occurrence, frequency, severity, progression, or duration of one or more symptoms or underlying causes or consequences of a condition, disease or symptom in the subject, such as one or more adverse symptoms, disorders, illnesses, pathologies, diseases, or complications caused by or associated with a disease or condition. Treatment methods affecting one or more underlying causes of the condition, disease or symptom are therefore considered to be beneficial. Stabilizing a disorder or condition is also a successful treatment outcome.

A therapeutic benefit or improvement therefore need not be complete ablation of any one, most or all symptoms, complications, consequences or underlying causes associated with the condition or disease. Thus, a satisfactory endpoint is achieved when there is an incremental improvement in a subject's condition, or a partial decrease, reduction, inhibition, suppression, limit, control or prevention in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, such as one or more adverse symptoms, disorders, illnesses, pathologies, diseases, or complications caused by or associated with the disease or condition, over a short or long duration of time (hours, days, weeks, months, etc.).

The terms “acceptable,” “effective,” or “sufficient” when used to describe the selection of any components, ranges, dose forms, etc. disclosed herein intend that said component, range, dose form, etc. is suitable for the disclosed purpose.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human.

### EXAMPLES

These examples are provided for illustrative purposes only and not to limit the scope of the claims provided herein.

### Example 1

### Preparation of CAR-T Cells

For transduction of MSGV γ-retroviral vector, plasmids were transfected using Lipofectamine 3000 (Thermo Fisher Scientific, MA, USA) along with pAmpho vector into GP2-293 packaging cell lines to generate viral supernatants. The supernatants were collected 48 hours after transfection, and virus binding plates were prepared by centrifugation on Retronectin (Takarabio, Shiga, Japan)-coated plates. Peripheral blood mononuclear cells (PBMCs) were activated by solidified anti-human CD3 Ab (OKT3, 5 μg/mL), and cultured in medium containing recombinant human IL-2 (400 IU/mL) for 3 days. PBMCs were added onto virus binding plates and cultured for 24 hours for 1^{st} infection. Cultured PBMCs were then transfer to another virus binding plates for 2^{nd} infection, and after 4 hours incubation, infected cells were expanded with fresh medium containing 400 IU/mL recombinant human IL-2 for 3 days. For transduction of SFG γ-retroviral vector, plasmids were transfected into PhoenixAmpho packaging cell lines using FuGENE® HD Transfection Reagent (Promega Corp., WI, USA), along with gag/pol genes (Cell Biolabs Inc., CA, US) and VSV-G vector (Takarabio, Shiga, Japan). T cells were isolated from PBMCs using EasySep™ Human T Cell Isolation Kit (Stem Cell Technologies, BC, Canada), and cultured with T Cell TransAct (Miltenyi Biotech, Bergisch Gladbach, Germany) and 10 ng/mL recombinant human IL-2 (Miltenyi, Bergisch Gladbach, Germany) for 2 days. T cells were kept culture in 10 ng/mL recombinant human IL-2 containing medium, and after 3 days from isolation, T cells were transduced with viral supernatant for 5 hours on plates coated with 20 μg/mL Retronectin. Transduced T cells were expanded using G-Rex (WilsonWolf, MN, USA) for 4 days. Transduction efficiencies were determined by flow cytometry. Either X-VIVO™ 15 Medium (Lonza, Basel, Switzerland) or CTS™ OpTmizer™ T Cell Expansion SFM (Thermo Fisher Scientific, MA, USA) were used as culture medium.

### Evaluation of In Vitro Tumor Cytotoxic Activity of CAR-T Cells

The in vitro target tumor cell killing activity of CAR-T cells was evaluated against human MSLN expressing cells. Untransduced (UTD) T cells were evaluated in parallel as a control article. The target cells used in this study were Capan-2 cells and MSTO-211H-Luc cells that endogenously expresses human MSLN. The target cells were seeded followed by the addition of CAR-T cells or UTD T cells that were diluted to acquire a 6-point series of Effector (CAR positive):Target cell (E:T) ratios: 3:1, 1:1, 0.3:1, 0.1:1, 0.03:1, and 0.01:1. After 48 hours of co-culture, viability of the target cell lines was measured with CellTiter-Glo® Luminescent Cell Viability Assay (Promega Corp., WI, USA) after washing out of T cells. The relative killing activity was calculated by comparing cell viability of the target cells in T cell co-cultured conditions with that in non-T cell co-cultured conditions.

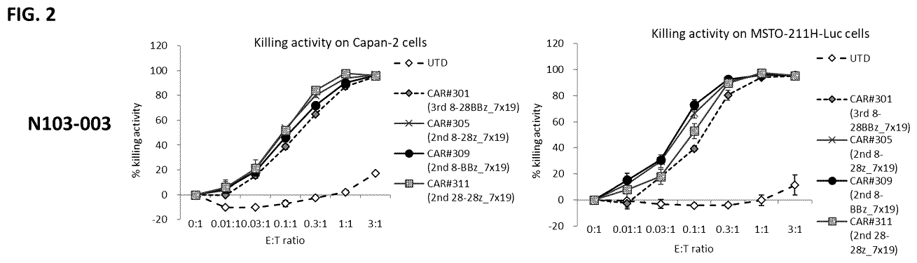

The in vitro target cell killing activities of CAR-T cells and UTD T cells against Capan-2, and MSTO-211H-Luc cells are presented in FIG. 2. All tested CAR-T cells demonstrated dose-dependent increase in target cell killing activity against Capan-2 and MSTO-211H-Luc cells. In contrast, UTD T cells showed limited ability to kill Capan-2, and MSTO-211H-Luc cells even at the highest E:T ratio. 2^{nd} 8-28z_7×19 CAR-T (CAR #305), 2^{nd} 8-BBz_7×19 CAR-T (CAR #309) and 2^{nd} 28-28z_7×19 CAR-T (CAR #311) cells demonstrated similar to higher target cell killing activity compared to 3rd 8-28BBz_7×19 CAR-T cells (CAR #301).

The description of the anti-MSLN CAR-IL-7-CCL19 constructs used in the experiment is described below:

CAR #3⁰¹ (3^{rd} 8-28BBz_7×19 with F2A, pMSGV)

Anti-MSLN CAR DNA fragment encoding anti-MSLN scFv, hinge and transmembrane region of human CD8, intracellular signal domain of human CD8, CD28, 4-1BB and CD3z

CAR #305 (2^{nd} 8-28z_7×19 with F2A, pMSGV)

Anti-MSLN CAR DNA fragment encoding anti-MSLN scFv, hinge and transmembrane region of human CD8, intracellular signal domain of human CD28 and CD3z

CAR #309 (2^{nd} 8-BBz_7×19 with F2A, pMSGV)

Anti-MSLN CAR DNA fragment encoding anti-MSLN scFv, hinge and transmembrane region of human CD8, intracellular signal domain of human 4-1BB and CD3z

CAR #311 (2^{nd} 28-28z_7×19 with F2A, pMSGV)

Anti-MSLN CAR DNA fragment encoding anti-MSLN scFv, hinge and transmembrane region of human CD28, intracellular signal domain of human CD28 and CD3z

CAR #3³⁴ (3rd 8-28BBz_7×19 with F2A, pSFG)

Anti-MSLN CAR DNA fragment encoding anti-MSLN scFv, hinge and transmembrane region of human CD8, intracellular signal domain of human CD8, CD28, 4-1BB and CD3z

CAR #3¹⁴ (2nd 8-28z_7×19 with F2A, pSFG)

Anti-MSLN CAR DNA fragment encoding anti-MSLN scFv, hinge and transmembrane region of human CD8, intracellular signal domain of human CD28 and CD3z

CAR #318 (2^{nd} 8-BBz_7×19 with F2A, pSFG)

Anti-MSLN CAR DNA fragment encoding anti-MSLN scFv, hinge and transmembrane region of human CD8, intracellular signal domain of human 4-1BB and CD3z

CAR #3²³ (2nd 28-28z_7×19 with F2A, pSFG)

Anti-MSLN CAR DNA fragment encoding anti-MSLN scFv, hinge and transmembrane region of human CD28, intracellular signal domain of human CD28 and CD3z

CAR #3⁴⁵ (2nd 28-28z_7×19 with P2A, pSFG)

Anti-MSLN CAR DNA fragment encoding anti-MSLN scFv, hinge and transmembrane region of human CD28, intracellular signal domain of human CD28 and CD3z

CAR #3⁴⁷ (2nd 8-28z_7×19 with P2A, pSFG)

Anti-MSLN CAR DNA fragment encoding anti-MSLN scFv, hinge and transmembrane region of human CD8, intracellular signal domain of human CD28 and CD3z

CAR #348 (3^{rd} 8-28BBz_7×19 with P2A, pSFG)

Anti-MSLN CAR DNA fragment encoding anti-MSLN scFv, hinge and transmembrane region of human CD8, intracellular signal domain of human CD8, CD28, 4-1BB and CD3z

CAR #349 (2^{nd} 8-BBz_7×19 with P2A, pSFG)

Anti-MSLN CAR DNA fragment encoding anti-MSLN scFv, hinge and transmembrane region of human CD8, intracellular signal domain of human 4-1BB and CD3z

CAR #357 (2^{nd} 8-BBz_7×19 with F2A, pSFG)

Anti-MSLN CAR DNA fragment encoding anti-MSLN scFv, hinge and transmembrane region of human CD8, intracellular signal domain of human 4-1BB and CD3z

CAR #358 (2^{nd} 8-BBz_7×19 with T2A, pSFG)

Anti-MSLN CAR DNA fragment encoding anti-MSLN scFv, hinge and transmembrane region of human CD8, intracellular signal domain of human 4-1BB and CD3z

CAR #3⁶⁴ (2nd 28-28z_7×19 with Non-Identical Nucleotide P2A Sequence, pSFG)

Anti-MSLN CAR DNA fragment encoding anti-MSLN scFv, hinge and transmembrane region of human CD28, intracellular signal domain of human CD28 and CD3z

CAR #365 (2^{nd} 8-BBz_7×19 with Non-Identical Nucleotide P2A Sequence, pSFG)

Anti-MSLN CAR DNA fragment encoding anti-MSLN scFv, hinge and transmembrane region of human CD8, intracellular signal domain of human 4-1BB and CD3z

In the constructs used in the experiment described above, GSG (peptide linker) was added to the N-terminus of each P2A, F2A, T2A sequence

### Evaluation of In Vivo Anti-Tumor Activity of CAR-T Cells

Female NSG mice were inoculated subcutaneously with 2 million (M) cells of MSLN-positive Capan-2 tumor cells. On day 7 after inoculation, CAR-T cells were administrated single intravenously with several doses (0.8 M, 2 M and 5 M for 3rd 8-28BBz_7×19 CAR-T (CAR #334), 3.2 M for 2^{nd} 8-28z_7×19 (CAR #314) and 2^{nd} 8-BBz_7×19 CAR-T (CAR #318), 5M for 2^{nd} 28-28z_7×19 CAR-T cells (CAR #323) as CAR positive cell number). As a control group, the vehicle control phosphate-buffered saline (PBS), or control UTD T cells was administered. The tumor volume (TV) of each mice was measured twice weekly.

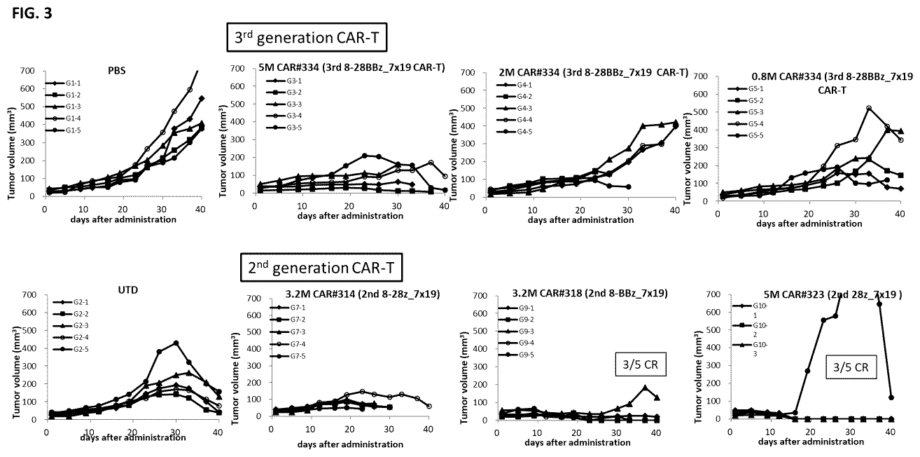

Tumor volume (TV) of mice in each treatment group (5 mice per group) are presented in FIG. 3. In 5 M 3rd 8-28BBz_7×19 CAR-T (CAR #334) group and 3.2 M 2^{nd} 8-28z_7×19 CAR-T group (CAR #314), TV tended to increase slowly in the first 3 weeks after administration, and tendency of mild decrease of TV was observed. On the other hand, in 5 M 2^{nd} 28-28z_7×19 CAR-T group (CAR #323) and 3.2 M 2^{nd} 8-BBz_7×19 CAR-T (CAR #318) group, tumor shrinkage was observed within 2 weeks after CAR-T treatment and 3 out of 5 mice in each group demonstrated the extinction of tumor tissues, suggesting the complete response (CR). These results indicated that 2^{nd} generation CAR-T armored with IL-7 and CCL19 has higher anti-tumor efficacy compared to 3^{rd} 8-28BBz_7×19 CAR-T construct.

### Evaluation of Anti-Tumor Activity of CAR-T Cells Using Histopathological Assessment of Xenografted Tumor Tissues

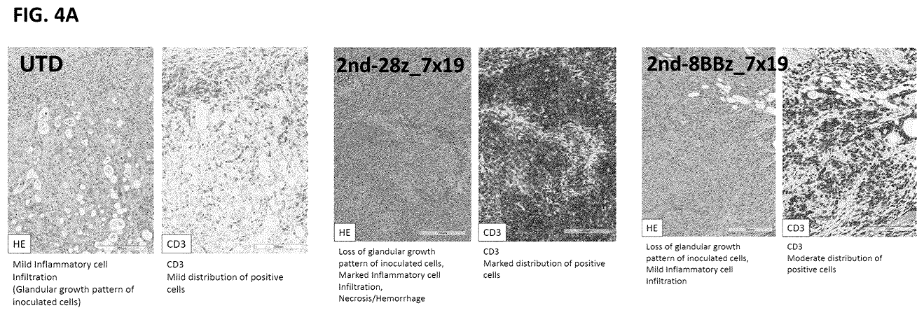

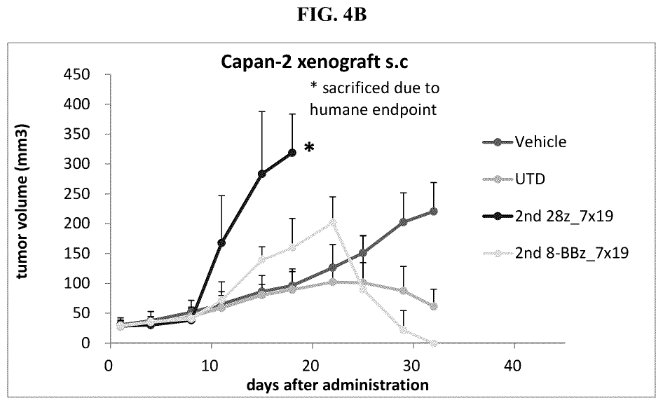

Female NSG mice were inoculated subcutaneously with 2 million (M) cells of mesothelin-positive Capan-2 tumor cells. On day 7 after inoculation, 5 M CAR positive cells of CAR-T cells were administrated single intravenously. Tested constructs were 2^{nd} 8-BBz_7×19 CAR-T (CAR #349) and 2^{nd} 28-28z_7×19 CAR-T cells (CAR #345). As a control group, the vehicle control phosphate-buffered saline (PBS), or equivalent total T cell numbers of control UTD T cells was administered. The tumor volume (TV) of each mice was measured twice weekly. At the endpoint of each group (Day 18 for 2^{nd} 28-28z_7×19 CAR-T (CAR #345) group and Day 32 for UTD or 2^{nd} 8-BBz_7×19 CAR-T (CAR #349) group), tumor xenografts were collected and microscopic examination of tissue slides were performed. See FIG. 4A.

Mean tumor volume (TV) of each group was plotted in FIG. 4B. 2^{nd} 28-28z_7×19 CAR-T (CAR #345) treated group demonstrated the acute increase of mean TV from 8 days after CAR-T administration and all mice were sacrificed on Day 18 due to GvHD-like symptoms which was assessed as humane endpoint. On the other hand, increase of mean TV was observed from Day 15 to Day 22 in 2^{nd} 8-BBz_7×19 CAR-T (CAR #349) treated group and these swelled tumor xenografts were rapidly diminished by Day 32. On Day 32, 3 out of 5 mice demonstrated the extinction of tumor, suggesting the complete response of 2^{nd} 8-BBz_7×19 CAR-T cells (CAR #349). Tumor xenografts treated with UTD T cells remained the glandular pattern of tumor cells and mild infiltration of human CD3 positive T cells were observed. Marked infiltration of human CD3 positive T cells were observed in tumor xenografts of 2^{nd} 28-28z_7×19 CAR-T (CAR #345) treated group. Collected tumor xenografts from 2^{nd} 8-BBz_7×19 CAR-T (CAR #349) treated mice showed that the tumors had shrunk at the endpoint, however, moderate infiltration of human CD3 positive T cells were confirmed in the remaining xenografts. In these xenografted tissues of CAR-T treated mice, glandular growth pattern of tumor cells was lost and tissues were filled with the infiltrated human T cells, which was totally different from that of UTD T cells treated mice. These results indicated that increase of tumor mass observed in CAR-T treated mice was result of the accumulation of administrated human T cells in the xenografted tumor microenvironment, which could be considered as pseudoprogression of tumor, suggesting the MOA-driven sign of efficacy resulted from IL-7-dependent T cell proliferation and CCL19-dependent accumulation of T cells.

### Evaluation of Anti-Tumor Activity of CAR-T Cells Using Bioluminescence Imaging (BLI) of Tumor Cells

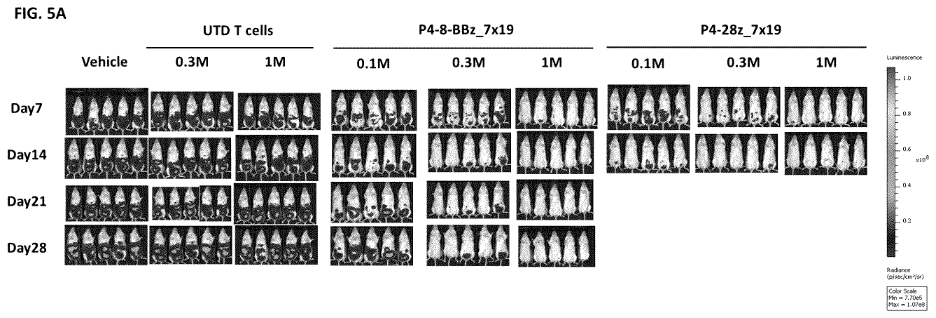

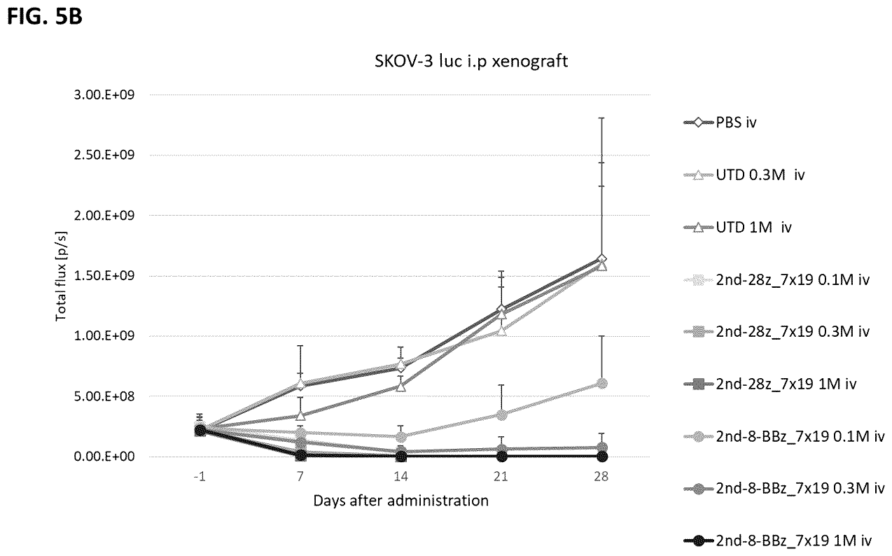

Mean tumor volume is a common means of evaluating tumor burden, yet effects measured by tumor volume (TV) can be complicated when the test article stimulates immune cell accumulation in tumor tissue, as would be expected with the use of CAR-T or other similar test articles. In vivo evaluation of tumor volume using a luciferase-expressing tumor cell line can be a more specific measure of antitumor efficacy in these models, as a decrease of tumor cells can be specifically measured despite no observed change or increases in tumor volume due to inflammation. Female NSG mice were inoculated intraperitoneally with 5 million (M) cells of MSLN-positive SKOV3-luc tumor cells. On day 4 after inoculation, CAR-T cells were administrated single intravenously with several doses (0.1 M, 0.3 M and 1 M for 2^{nd} 8-BBz_7×19 CAR-T (CAR #365) and 2^{nd} 28-28z_7×19 CAR-T cells (CAR #364) as CAR positive cell number). As a control group, the vehicle control phosphate-buffered saline (PBS), or equivalent total T cell numbers of control UTD T cells was administered for 0.3 M and 1 M UTD groups. The mice were monitored through 28 days. Total flux (TF), which is a measure of the luminescence of SKOV3-luc cells, is proportional to the number of SKOV3-luc tumor cells present in the animal and can be used to evaluate anti-tumor efficacy, was measured once per week. See FIG. 5A.

The duration of this study was 28 days, however, all 2^{nd} 28-28z_7×19 CAR-T (CAR #364) treated groups with 0.1 M, 0.3 M and 1 M dose was sacrificed on Day 14 post CAR-T injection due to humane endpoint. Both CAR-T treated groups (CAR #364 and CAR #365) with a dose of 1M CAR-T cells demonstrated a reduction in mean total flux at Day 14, which was initially observed upon the first measurement at Day 7 post CAR-T injection. The reduction of mean TF was sustained for the duration of the study period in 2^{nd} 8-BBz_7×19 CAR-T (CAR #365) treated group at 0.3 M and 1 M doses. In 0.1 M dose group of 2^{nd} 8-BBz_7×19 (CAR #365) CAR-T, tumor cells were remained to be present throughout the study duration. TF of vehicle control group and both dose groups of UTD T cells was continuously increased from Day 7 to the endpoint. These results indicate evidence of dose-dependent anti-tumor activity of both 2^{nd} 8-BBz_7×19 CAR-T (CAR #365) and 2^{nd} 28-28z_7×19 CAR-T (CAR #364) cells in NSG mice bearing SKOV3-luc xenografts as measured by significant reduction of mean TF, a measure of luminescence. See FIG. 5B.

To evaluate the antigen-independent in vivo cell expansion capacity of CAR-T cells, body weight (BW) change in mice with or without antigen-expressing tumors were investigated after CAR-T administration. BW decrease was typically observed as the Graft-versus host disease (GvHD) symptom after human T cell injection into mice.

Female NSG mice were inoculated subcutaneously with 2 million (M) cells of MSLN-positive Capan-2 tumor cells. On day 7 after inoculation, 3 M CAR positive cells of CAR-T cells were administrated single intravenously. Tested CAR-T constructs were 2^{nd} 8-28z_7×19 (CAR #347), 2^{nd} 8-BBz_7×19 CAR-T (CAR #349), and 2^{nd} 28-28z_7×19 CAR-T cells (CAR #345). As a control group, equivalent total cell numbers of UTD T cells (4.4 M cells) was administered. These T cells were administrated into non tumor bearing mice on the same day. Body weight (BW) of each mice was measured twice weekly.

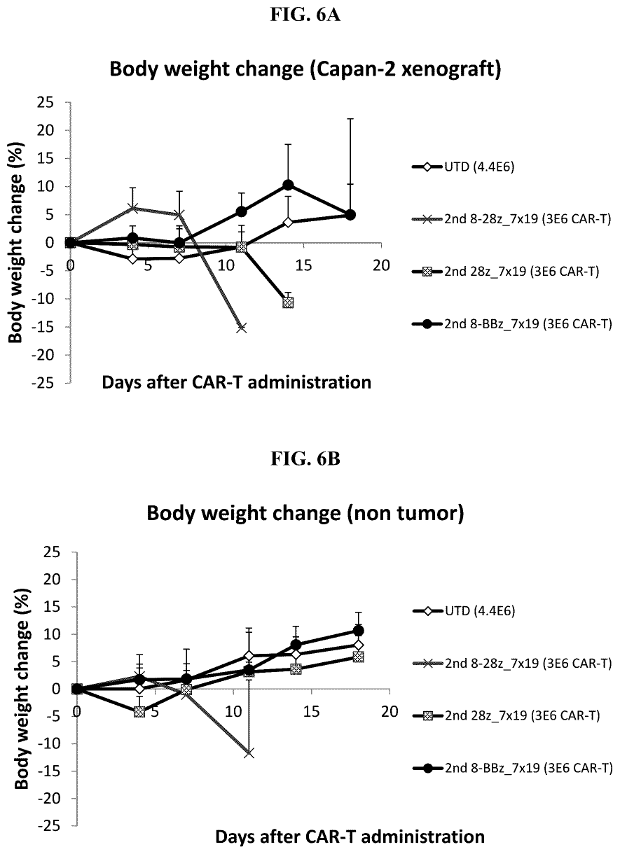

Mean BW change (%) of each mice group with 5 animals was plotted in FIG. 6A and FIG. 6B.

In Capan-2 tumor xenografted model, acute BW decrease was observed 11 days after administration of 2^{nd} 8-28z_7×19 CAR-T cells (CAR #347). BW decrease was also observed 14 days after 2^{nd} 28-28z_7×19 CAR-T cells (CAR #345) administration, whereas significant BW decrease was not observed in UTD T cells nor in 2^{nd} 8-BBz_7×19 CAR-T (CAR #349) treated group. In non-tumor bearing condition, significant decrease of mean BW was observed at Day 11 only with 2^{nd} 8-28z_7×19 CAR-T (CAR #347) treated group and not with the other CAR-T treated groups and UTD group, suggesting the high capacity of in vivo expansion of 2^{nd} 8-28z_7×19 CAR-T (CAR #347) without antigen-stimulation which can be considered as the off-target toxicity risk.

### In Vivo Safety Assessment of CAR-T Cells

Safety of construct CAR #365 (2^{nd} 8-BBz_7×19), CAR #364 (2^{nd} 28-28z_7×19), and untransduced (UTD) cells were evaluated after a single dose administration of 7.5×10⁶ total cells (equivalent to 3M CAR+ cells) in nontumor-bearing female NSG mice. In this study, animals were humanely euthanized 17 days post CAR-T administration and evaluated for changes in serum chemistry, organ weight, and macroscopic and microscopic pathology.

There were no significant changes in serum chemistry or organ weights among the three groups, and all animals survived to terminal necropsy. Macroscopic findings were present in animals administered CAR #364 and consisted of lung discoloration and spleen enlargement, both of which correlated to minimally increased organ weights and microscopically to the presence of the CAR-T cells in these tissues, i.e. mixed cell inflammation (lungs) and increased cellularity of white pulp (spleen).

UTD cells had minimal presumptive CAR-T infiltration/inflammation in the lung and spleen. This finding is considered possibly related to Graft versus Host Disease (GvHD) as the pattern in the lung was typical. Animals administered CAR #365 were similar to those administered UTD cells with a lower incidence of lung mononuclear infiltrates or mixed cell inflammation, and a higher incidence of presumptive CAR-T cells engrafting in the spleen, and additionally in the bone marrow. Additional findings were present in one animal each in the liver (minimal presumptive CAR-T infiltrates possibly consistent with GvHD).

Animals administered CAR #364 had a higher severity of findings as those administered UTD cells, with additional findings in the liver. These findings are suggestive of tonic signaling resulting in exacerbated effects of GvHD and/or other mechanisms of CAR-T activation associated with the cytokine armoring.

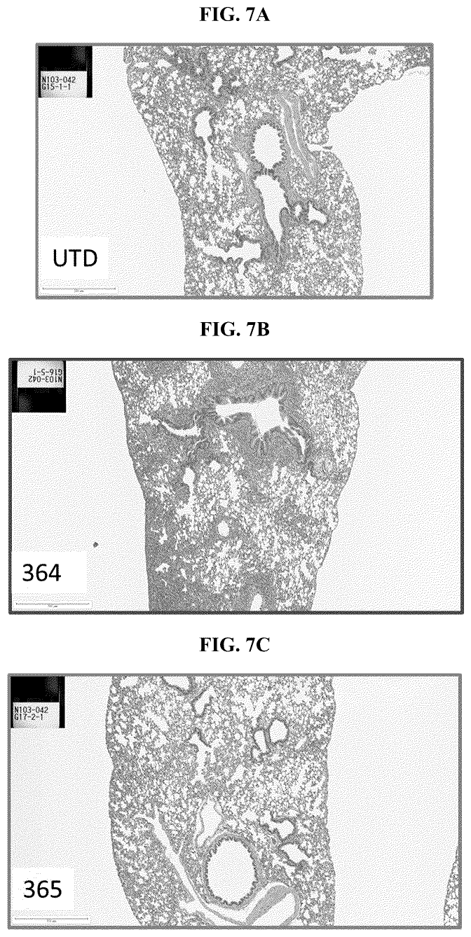

Overall, construct CAR #365 was well tolerated in nontumor-bearing female NSG mice with minimal findings similar to the UTD cells which indicates the findings may be related to GvHD. Construct CAR #365 also displayed a superior safety profile in comparison to construct CAR #364, which had signs of uncontrolled cellular proliferation in normal tissues. See FIGS. 7A-7C.

### Flow Cytometry Analysis of Administrated T Cells in Capan-2 Xenografted NSG Mice

Female NOG MHC Class I/II KO mice were inoculated subcutaneously with 2 million (M) cells of mesothelin-positive Capan-2 tumor cells. On day 7 post tumor inoculation, 5 M CAR positive cells of CAR-T cells were administrated single intravenously. Tested CAR-T constructs were 2^{nd} 8-BBz_7×19 CAR-T (CAR #364) and 2^{nd} 28-28z_7×19 CAR-T cells (CAR #365). As a control group, equivalent total cell numbers of UTD T cells (12.5 M cells) was administered. Blood, spleen and xenografted tumor tissues were collected on Day 13, 27 and 41 post CAR-T injection for 2^{nd} 8-BBz_7×19 CAR-T (CAR #365) dose group. Blood and tissues were collected as well in 2^{nd} 28-28z_7×19 CAR-T (CAR #364) cells dose group on Day 13 post CAR-T injection because all animals in this group were sacrificed on Day 13 post CAR-T injection due to humane endpoint. For tumor sample, cells were collected using Tumor & Tissue Dissociation Reagent kit (TTDR, BD Biosciences). Spleen samples were dissociated and filtered to obtain cell suspension. The cells were incubated with his-tagged mesothelin in FACS buffer (500 mL DPBS−/5 ml NaN3/10 mL FBS) for 30 min at RT in the dark. Cells were washed and centrifuged followed by incubation with zombie NIR in PBS for 15 min at RT in the dark. Cells were incubated with antibody mixture (CD3/FITC, CD8/BV510, CTLA4/PE-Cy7, LAG-3/Alexa Fluor 647, PD-1/BV421, TIM-3/PerCP-Cy5.5, anti-his Ab/PE). Washed cells were incubated with BD FACS lysing buffer (BD Biosciences) in DW. After washing step, cells were resuspended in FACS buffer (500 mL DPBS−/5 ml NaN3/10 mL FBS) and analyzed with BD FACSLyric™ flow cytometer. FlowJo software (BD Biosciences) was used for data analysis.

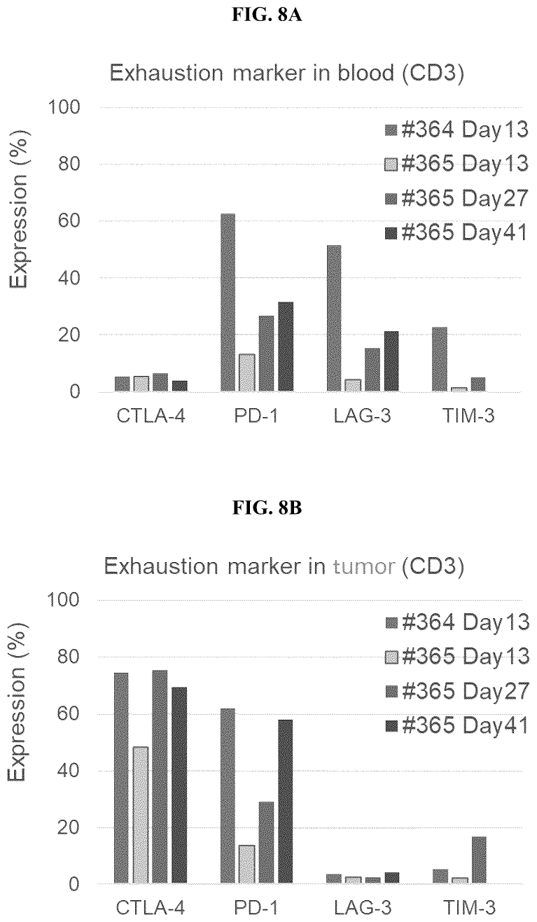

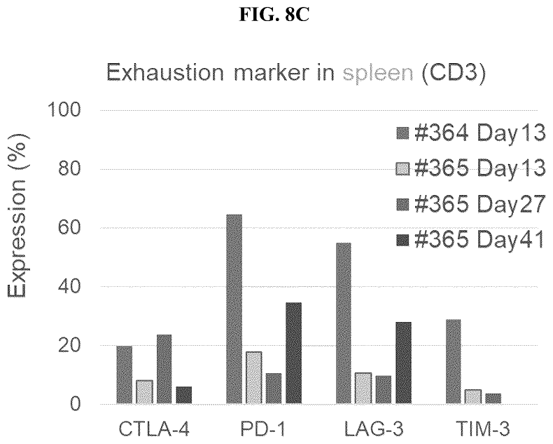

Exhaustion marker (CTLA4, PD-1, LAG-3 and TIM3) expression on CD3+ cells in blood, tumor and spleen are shown in FIGS. 8A-8C. In the peripheral (blood and spleen), CAR #365 showed less exhaustion marker expression than CAR #364. In tumor, LAG-3 and TIM-3 expression was low both in #364 and #365. For CTLA-4 and PD-1, CAR #365 showed slower expression than CAR #364.

### Evaluation of Soluble Human Mesothelin on CAR-T Activation

To evaluate the blocking activity of soluble form of MSLN on CAR-T function, 2^{nd} 8-BBz_7×19 CAR-T (CAR #349) and 2^{nd} 28-28z_7×19 CAR-T cells (CAR #345) were pre-incubated with soluble MSLN (sMSLN) with several doses (0.01, 0.03, 0.1, 0.3, and 1 ug/mL) for 24 hours respectively, and the washed CAR-T cells were incubated with MSLN-positive Capan-2 tumor cells. 48 hours after co-culture of tumors and CAR-T cells, co-culture supernatant was collected and human IFNγ secretion level was measured by ELISA.

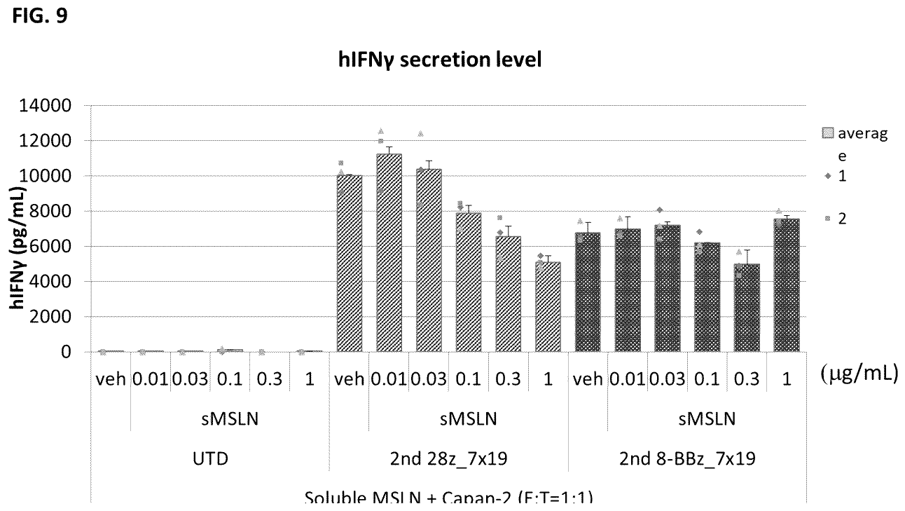

As shown in FIG. 9, around 10,000 pg/mL of human IFNγ was secreted from co-culture supernatant of Capan-2 cells and 2^{nd} 28-28z_7×19 CAR-T (CAR #345) cells pre-incubated with vehicle PBS. IFNγ secretion from 2^{nd} 8-BBz_7×19 CAR-T cells (CAR #349) was around 7,000 μg/mL in the same condition. IFNγ secretion from antigen-stimulated 2^{nd} 28-28z_7×19 CAR-T cells (CAR #345) was dose-dependently decreased by pre-incubation with sMSLN, whereas dose-dependent inhibition on IFNγ secretion from antigen-stimulated 2^{nd} 8-BBz_7×19 CAR-T cells (CAR #349) was not induced by sMSLN.

Comparison of Secretion Levels of IL-7 and CCL19 from CAR-T Cells

Secretion levels of IL-7 and CCL19 were evaluated by ELISA using the culture supernatant collected from cultured 2^{nd} 8-BBz_7×19 CAR-T (with P2A, CAR #349) cells, 2^{nd} 8-BBz_7×19 CAR-T (with F2A, CAR #357) cells and 2^{nd} 8-BBz_7×19 CAR-T (with T2A, CAR #358) cells. Quantification of IL-7-CCL19 fusion protein secreted from each CAR-T cells was performed using MSD system.

MSD GOLD 96-well Streptavidin SECTOR Plate (MSD Cat. No.: L15SA-5) was incubated with 250 uL/well 1×PBST containing 3% BSA for >30 minutes. Plates were washed 3 times with 1×PBST and was blotted to remove excess buffer (herein after called as washing step). The Biotin Anti-human MIP-30 Antibody from the U-PLEX Human MIP-3β Antibody Set (MSD, Cat. No.: B21VA-3) was used as capture reagent. 25 uL/well 1× capture antibody was added and incubated at room temperature for 1 hour. After washing step, the sample was diluted with equal volume of assay buffer (1×PBST containing 3% BSA). 50 uL/well of 2× diluted samples was added onto the plate and incubated at room temperature for 1.5 hour with gentle shaking on a shaker. Followed by washing step, 1× detection antibody solution was prepared by diluting the detection antibody from the U-PLEX Human IL-7 Antibody Set (MSD Cat. No.: B21UP-3) 100 fold with the assay buffer (1×PBST containing 3% BSA). 50 uL/well of 1× detection antibody solution was added and incubated at room temperature for 1.5 hour with gentle shaking on a shaker. After the washing step, 150/uL of 2×MSD Reading Buffer was added and the plate was read immediately on a MSD plate reader.

Secretion levels of IL-7, CCL19 and IL-7/CCL19 fusion protein from culture supernatant of each CAR-T cells were indicated in Table 2. 2^{nd} 8-BBz_7×19 CAR-T (with P2A, CAR #349) cells demonstrated the highest expression levels of IL-7 and CCL19 from the same amount of the culture supernatant. 2^{nd} 8-BBz_7×19 CAR-T (with P2A, #349) showed the highest cleaved IL-7 and CCL19 concentration. For IL-7/CCL19 fusion protein, ratio of cleaved/uncleaved fusion protein of P2A construct is comparable to that of 2^{nd} 8-BBz_7×19 CAR-T (with T2A, #358) and lower than that of 2^{nd} 8-BBz_7×19 CAR-T (with F2A, #357).

TABLE 2
Concentration of IL-7, CCL19 and IL-7/CCL19 fusion protein
and those ratio in the culture supernatant of CAR-T cells.

| | | IL-7 | CCL19 | IL7-CCL19 | Fusion/ | Fusion/ |
|---|---|---|---|---|---|---|
| | | conc. | conc. | fusion conc. | IL-7 | CCL19 |
| ID | 2A | (pg/mL) | (pg/mL) | (pg/mL) | ratio | ratio |
| #349 | P2A | 7255 | 7690 | 152 | 2.1% | 2.0% |
| #357 | F2A | 2516 | 5091 | 386 | 15.3% | 7.6% |
| #358 | T2A | 4203 | 5304 | 72 | 1.7% | 1.4% |

Evaluation of In Vivo Anti-Tumor Activity of 2^{nd} Generation And 3^{rd} Generation CAR-T Cells Using Bioluminescence Imaging of Tumor Cells

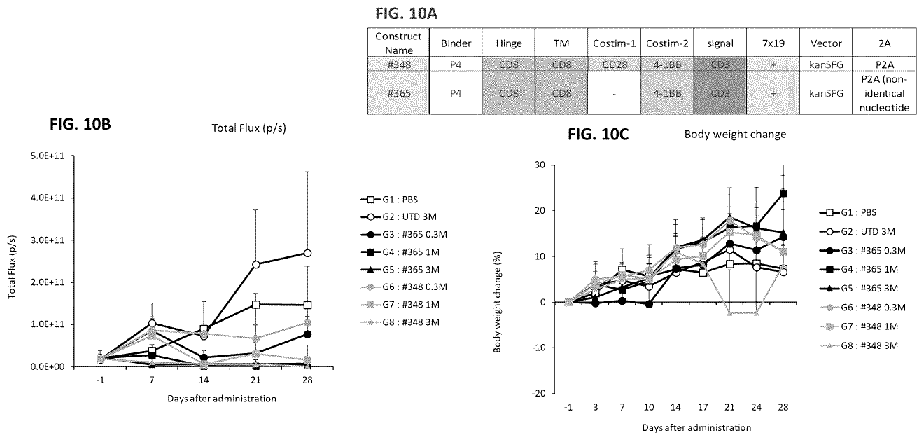

In order to fairly compare the efficacy of 3^{rd} generation 7×19 CAR-T and 2nd generation 7×19 CAR-T, 3rd 8-28BBz_7×19 CAR-T (CAR #348) and 2^{nd} 8-BBz_7×19 CAR-T (CAR #365) cells were generated using the same retroviral vector components including P2A peptide sequences, as shown in FIG. 10A. Female NSG mice were inoculated subcutaneously with 5 million (M) cells of mesothelin-positive HepG2-RedFluc cells. On day 7 after inoculation, 3rd 8-28BBz_7×19 CAR-T (CAR #348) and 2nd 8-BBz_7×19 CAR-T (CAR #365) cells were administered single intravenously with several doses (0.3M, 1M and 3M as CAR positive cell number). As a control group, the vehicle control phosphate-buffered saline (PBS), or equivalent total T cell numbers of control untransduced (UTD) T cells to 3M CAR positive cells was administered for UTD 3M group. Total flux (TF), which is a measure of the luminescence of HepG2-RedFluc cells proportional to the number of tumor cells present in the animal, was measured once per week for evaluation of anti-tumor efficacy of CAR-T cells.

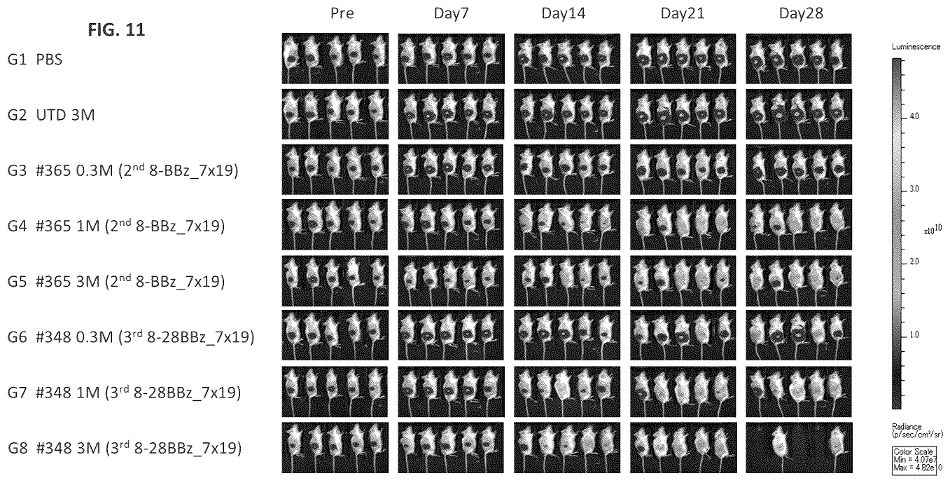

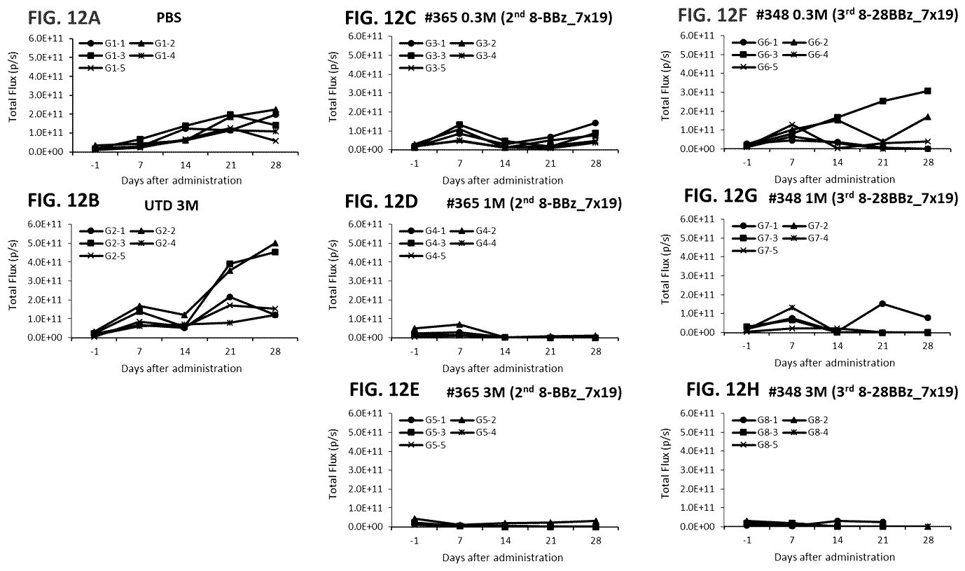

Both 2nd 8-BBz_7×19 CAR-T (CAR #365) and 3rd 8-28-BBz_7×19 CAR-T (CAR #348) demonstrated a reduction in mean total flux in a dose dependent manner. In 3M CAR-T group, both groups showed comparable efficacy, but 3 out of 5 mice in 3rd 8-28-BBz_7×19 CAR-T (CAR #348) group was sacrificed by the end of the study due to the humane endpoint. Significant decrease of TF was also observed with both of 1M CAR-T groups by the endpoint whereas 1 out of 5 mice f° m 3rd 8-28-BBz_7×19 CAR-T (CAR #348) had tumor regrowth after day 14. In 0.3 M CAR-T group, comparable level of TF decrease was observed with both CAR-T groups at the endpoint, however, 2nd 8-BBz_7×19 CAR-T (CAR #365) had shown better tumor control in all tested mice, whereas 1 out of 5 tested mice f° m 3rd 8-28-BBz_7×19 CAR-T (CAR #348) group did not demonstrate tumor regression throughout the study period. These results indicate 2nd 8-BBz_7×19 CART (CAR #365) has higher and prolonged anti-tumor activity compared to 3rd 8-28BBz_7×19 CART (CAR #348). See FIG. 10A-B, FIG. 11, and FIG. 12A-H.

### Example 2

An Open-Label, Dose Escalation, Phase 1, First-In-Human Study of Modified Immune Cells Expressing a CAR Described Herein, IL-7 and CCL19 in Adult Patients with Mesothelin-Expressing Advanced or Metastatic Solid Tumors

### Study Design:

This is an open-label, nonrandomized, phase 1, first-in-human study to evaluate the safety and tolerability of modified immune cells expressing a CAR described herein, IL-7, and CCL19 administered intravenously (IV) in patients with mesothelin-expressing advanced or metastatic solid tumors with no standard therapeutic alternative with established clinical benefit. Ovarian cancer, mesothelioma, Gastric cancer and non-small lung cell cancer (NSCLC) are the high priority target population for the modified immune cells expressing a CAR described herein, IL-7, and CCL19. Patients with other cancer types will be enrolled based on the discussion between the investigator(s) and the sponsor. Up to 21 DLT evaluable patients will be treated with the modified immune cells expressing a CAR described herein, IL-7, and CCL19 in 1 of the 5 proposed dosing cohorts. Dose escalation will be guided by the Bayesian Optimal Interval (BOIN) design which is based on the observed DLT rate for each dose level. Dose escalation/de-escalation decisions will be determined within the recommended dose by BOIN, taking into account safety other than DLTs, efficacy and cellular kinetics (CK), and be made jointly by the sponsor and the investigator at meetings, including the end-of-cohort meetings. Additional emerging translational data (eg, soluble mesothelin, cancer antigen 125 [CA125]) may also be used, when feasible, to aid decision-making of dosing additional patients at a given dose level. Recommended phase 2 dose (RP2D) will be determined based on aggregated observations of safety, efficacy, CK and biomarker assessments at each dose-level. In any dose cohort, infusion of the modified immune cells expressing a CAR described herein, IL-7, and CCL19 of the first and second patient will be separated by 14 (or more) days. The second and subsequent patients may be dosed concurrently. Dose cohorts will be separated by a minimum of 28 days (from the last infusion in the precedent cohort to the first infusion in the subsequent cohort). Toxicity will be evaluated according to the National Cancer Institute Common Terminology Criteria for Adverse Events (NCI CTCAE), Version 5.0. Cytokine release syndrome (CRS) and immune effector cell-associated neurotoxicity syndrome (ICANS) will be evaluated according to American Society for Transplantation and Cellular Therapy (ASTCT) consensus. For monitoring, grading, and management of toxicities associated with immune effector cell therapies, CARTOX (CAR-T-cell-therapy-associated TOXicity) recommendations will be used.

This study consists of the Prescreening, Screening, Pretreatment, Treatment and Primary Follow-up, and Secondary Follow-up phases. The Prescreening phase may begin on the date the patient signs the institutional review board/independent ethics committee (IRB/IEC)-approved informed consent form (ICF) for the assessment of mesothelin expression in tumor cells. The Screening phase starts with the date when one of procedures in the Screening phase take place. The patient is provided with a study-specific patient number on the date of Prescreening ICF or the date of Main ICF, whichever comes first. The Screening phase includes confirmation of eligibility and enrollment into the study, and ends with the start of leukapheresis procedures. The Pretreatment phase is defined as the period from the start of leukapheresis procedures through the completion of conditioning chemotherapy until the start of infusion. During the Pretreatment phase, leukapheresis, bridging therapy, and conditioning chemotherapy will be performed. The Treatment and Primary Follow-up phase begins with administration of the modified immune cells expressing a CAR described herein, IL-7, and CCL19 and continues up to Month 13. Secondary Follow-up phase begins with the end of the Treatment and Primary Follow-up period and continues up to Month 37. In the Pretreatment, the Treatment and Primary Follow-up and Secondary Follow-up phases, patients may complete study participation due to death, consent withdrawal, or other predefined situations.

Patients will be followed according to the visit schedule to ensure adequate data are collected for the proper assessment of study primary and secondary objectives. Patients may voluntarily withdraw from the Treatment and Primary Follow-up phase or be dropped from it at the discretion of the investigator at any time. It is anticipated that patients may leave the Primary Follow-up and move to Secondary Follow-up due to reasons including:
Disease progression
Patient voluntary withdrawal from the Primary Follow-up

Patients who discontinue the Treatment and Primary Follow-up phase before Month 13 will continue to be followed in the Secondary Follow-up phase in order to collect data requested from health authority (eg. protocol defined AEs) up to 3 years after the infusion of the modified immune cells expressing a CAR described herein, IL-7, and CCL19.

The first visit in the Secondary Follow-up phase is determined according to the time when the patient discontinued in the Treatment and Primary Follow-up phase. For example, if the patient discontinues from the Treatment and Primary Follow-up phase at Month 7, the first visit in the Secondary Follow-up phase will be Month 10.

### Primary Objectives:

To evaluate the safety and tolerability of the modified immune cells expressing a CAR described herein, IL-7, and CCL19.
To determine the RP2D of the modified immune cells expressing a CAR described herein, IL-7, and CCL19.

### Secondary Objectives:

To evaluate the antitumor activity of the modified immune cells expressing a CAR described herein, IL-7, and CCL19.
To characterize the CK of the modified immune cells expressing a CAR described herein, IL-7, and CCL19.
To determine the number and percent of embodiments with replication competent retrovirus (RCR)-positive test results.

### Subject Population:

Patients with mesothelin-expressing advanced or metastatic solid tumors. The study will include up to 21 DLT evaluable patients.

### Dose Levels:

The following cohorts with ascending dose levels are to be tested:
Cohort (−1): 0.3×10⁷ chimeric antigen receptor (CAR) (+) cells/body. (In case Cohort 1 is not tolerable)
Cohort 1: 1×10⁷ CAR (+) cells/body [starting dose].
Cohort 2: 1×10⁸ CAR (+) cells/body.
Cohort 3: 5×10⁸ CAR (+) cells/body.
Cohort 4: 1×10⁹ CAR (+) cells/body.
Route of Administration: Intravenous

### Duration of Treatment:

Single IV infusion. Conditioning chemotherapy precedes treatment. (Cohorts where conditioning chemotherapy is not administered may be tested.)

### Main Criteria for Inclusion:

1. Male or female patients aged≥20 years at the time of signing informed consent.
2. Histologically or cytologically confirmed advanced or metastatic solid tumors who have no option with or are intolerant of standard therapies with a proven clinical benefit.
3. Mesothelin-expression (≥50% positive on viable tumor cells with intensity of 2+ and/or 3+) must be determined on the tumor locally by immunohistochemistry using a validated assay, scoring and staining confirmed by the sponsor. Fresh biopsy sample must be used for eligibility assessment unless archived biopsy sample obtained within 6 months prior to leukapheresis procedures is available.
4. Life expectancy≥12 weeks.
5. Eastern Cooperative Oncology Group performance status of 0 or 1.
6. Adequate organ function as confirmed by clinical laboratory values as specified below:
a) Total bilirubin≤1.5× the upper limit of the normal range (ULN) except in patients with Gilbert's syndrome. Patients with Gilbert's syndrome may enroll with direct bilirubin≤3×ULN of the direct bilirubin. Elevated indirect bilirubin due to post-transfusion hemolysis is allowed.
b) Alanine aminotransferase (ALT) or aspartate aminotransferase (AST) must be <3×ULN. AST and ALT may be elevated up to 5×ULN if the elevation can be reasonably ascribed to the presence of metastatic disease in the liver.
c) Calculated creatinine clearance>50 mL/min (Cockcroft-Gault formula).
d) Hemoglobin must be ≥9 g/dL.
e) Neutrophil count must be >1000/mm³.
f) Absolute lymphocyte count must be >500/mm³.
g) Platelet count must be >75,000/mm³.
7. Patients must have radiographically measurable disease as defined by Response Evaluation Criteria in Solid Tumors, Version 1.1 (RECIST 1.1).
8. Female patients who:
a) Are postmenopausal (natural amenorrhea) for at least 1 year before the screening visit, OR
b) Are surgically sterile, OR
c) If they are of childbearing potential, agree to practice 1 highly effective nonhormonal method of contraception and 1 additional effective (barrier) method at the same time, from the time of signing the informed consent through at least 12 months following infusion of the modified immune cells expressing a CAR described herein, IL-7, and CCL19, OR
d) Agree to practice true abstinence, when this is in line with the preferred and usual lifestyle of the subject. Note: Periodic abstinence (eg, calendar, ovulation, symptothermal, postovulation methods), withdrawal, spermicides only, and lactational amenorrhea are not acceptable methods of contraception.
9. Male patients, even if surgically sterilized (ie, postvasectomy), who:
a) Agree to practice effective barrier contraception from the time of signing the informed consent through at least 12 months following infusion of the modified immune cells expressing a CAR described herein, IL-7, and CCL19, OR
b) Agree to practice true abstinence, when this is in line with the preferred and usual lifestyle of the subject. Note: Periodic abstinence (eg, calendar, ovulation, symptothermal, postovulation methods), withdrawal, spermicides only, and lactational amenorrhea are not acceptable methods of contraception.
10. Voluntary written consent must be given before performance of any study-related procedures not part of standard medical care, with the understanding that consent may be withdrawn by the patient at any time without prejudice to future medical care.
11. Willingness and ability to comply with scheduled visits and study procedures.

### Main Criteria for Exclusion:

1. Active systemic infections.
2. Known hepatitis B surface antigen (HBsAg) positive, or known or suspected active hepatitis C virus (HCV) infection. Patients who have positive hepatitis B core antibody (HBcAb) or hepatitis B surface antibody (HBsAb) can be enrolled but must have an undetectable hepatitis B virus (HBV) viral load. Patients who have positive hepatitis C virus antibody (HCVAb) must have an undetectable HCV viral load.
3. Coagulation disorders, or other major medical illnesses including, respiratory or immune system, and obstructive/restrictive pulmonary disease.
4. Patients with known cardiovascular and cardiopulmonary disease defined as unstable angina, clinically significant arrhythmia, myocardial infarction, congestive heart failure, Left Ventricular Ejection Fraction (LVEF)<45%, baseline oxygen saturation<93% on room air. A well-controlled atrial fibrillation would not be an exclusion whereas uncontrolled atrial fibrillation would be an exclusion.
5. Any serious medical or psychiatric illness that could, in the investigator's opinion, potentially interfere with the completion of treatment according to this protocol.
6. History of malignancy other than nonmelanoma skin cancer or carcinoma in situ (eg, cervix, bladder, breast) unless disease free for ≥3 years.
7. Any disease requiring systemic steroid treatment.
8. Any prior use of cell and gene therapy(ies).
9. Treatment with any investigational products (except for cell or gene therapy) within 14 days before leukapheresis procedures or 28 days before treatment with conditioning chemotherapy or the modified immune cells expressing a CAR described herein, IL-7, and CCL19.
10. Systemic anticancer therapy (including immuno-oncology therapies) and treatment with radiotherapy within 14 days before leukapheresis procedures or treatment with conditioning chemotherapy or the modified immune cells expressing a CAR described herein, IL-7, and CCL19.
11. Treatment with major surgery within 28 days before leukapheresis procedures or treatment with conditioning chemotherapy or the modified immune cells expressing a CAR described herein, IL-7, and CCL19 (minor surgical procedures such as catheter placement are not exclusionary criteria).
12. Previous treatment with any mesothelin-targeted therapy.
13. Any unresolved toxicity of Grade 3 or higher from previous anticancer therapy.
14. Patients with risk of bleeding as judged by the investigator.
15. Presence of central nervous system metastasis or other significant neurological conditions (patient with central nervous system metastases that have been effectively treated where necessary and stable can be enrolled).
16. Patients with human immunodeficiency virus (HIV) seropositive and/or human T-cell lymphotropic virus (HTLV) seropositive.
17. Patients with a history of organ transplantation or awaiting organ transplantation.
18. Patients with severe immediate hypersensitivity to any of the agents including cyclophosphamide, fludarabine, or streptomycin.
19. Admission or evidence of illicit drug use, drug abuse, or alcohol abuse.
20. Live vaccine≤6 weeks prior to start of conditioning regimen.
21. Female patients who are lactating and breastfeeding or have a positive serum pregnancy test (urine pregnancy test is allowed before treatment with conditioning chemotherapy and the modified immune cells expressing a CAR described herein, IL-7, and CCL19).

Note: Female patients who are lactating will be eligible if they discontinue breastfeeding before the treatment with the modified immune cells expressing a CAR described herein, IL-7, and CCL19.

### Main Criteria for Evaluation and Analyses:

### Primary Endpoints:

Incidence of dose-limiting toxicities (DLTs).

Incidence of treatment-emergent adverse events (TEAEs).

Incidence of adverse events (AEs) of clinical interest including severe ICANS, CRS, hemophagocytic lymphohistiocytosis, macrophage activation syndrome, and tumor lysis syndrome.

### Secondary Endpoints:

Overall response rate (ORR), disease control rate (DCR), duration of response (DOR), time to progression (TTP), and progression-free survival (PFS) as assessed by the investigator according to RECIST 1.1 and iRECIST.

Overall survival (OS).

CK-related parameters evaluated by CAR vector copy number (Cₘₐₓ [maximum (peak) observed in peripheral blood drug concentration after single dose administration], tₘₐₓ [time of first occurrence of maximum observed peripheral blood concentration], Cₗₐₛₜ [last observed quantifiable concentration in peripheral blood], tₗₐₛₜ [persistence: time of last observed quantifiable concentration in peripheral blood (days)]; AUC [area under the blood concentration-time curve]).

Number and percentage of embodiments with RCR-positive test results.

### Statistical Considerations:

The incidence and percentage of DLTs and TEAEs will be summarized by System Organ Class and Preferred Term of the International Council for Harmonisation (ICH) Medical Dictionary for Regulatory Activities (MedDRA) and by grade. In addition, the following events will be summarized with the same method.
Treatment-related TEAEs.
Grade 3 or higher TEAEs.
Grade 3 or higher treatment-related TEAEs.
Serious adverse events (related and regardless of relationship).

The AEs of clinical interest and RCR will be summarized by counts and percentages.

For the secondary efficacy endpoints, point estimate and the 2-sided 95% exact binominal confidence intervals will be computed for binary endpoints and time-to-event endpoints will be analyzed descriptively using Kaplan-Meier method.

CK parameters will be summarized using descriptive statistics. Individual concentration-time data and individual CK parameters will be presented in listings and tabulated using summary statistics by dose cohort. Individual and mean concentration-time profiles will be plotted by dose cohort.

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. An isolated nucleic acid molecule comprising:
a polynucleotide encoding a chimeric antigen receptor (CAR) comprising an antibody that specifically recognizes human mesothelin, a CD8 hinge region, a CD8 transmembrane region, a 4-1BB intracellular region, and a CD3ζ intracellular region;
a polynucleotide encoding IL-7; and
a polynucleotide encoding CCL19.

2.-3. (canceled)

4. The isolated nucleic acid molecule of claim 1, wherein the antibody comprises a heavy chain variable region (VH) and a light chain variable region (VL), wherein the VH comprises three complementarity-determining regions (CDRs) comprising SEQ ID NOs: 1-3, and wherein the VL comprises three CDRs comprising SEQ ID NOs: 4-6.

5. The isolated nucleic acid molecule of claim 4, wherein the VH comprises SEQ ID NO: 7 and the VL comprises SEQ ID NO: 8.

6. The isolated nucleic acid molecule of claim 1, wherein the antibody comprises a single-chain variable fragment (scFv) format.

7. The isolated nucleic acid molecule of claim 1, wherein the antibody comprises SEQ ID NO: 9.

8. The isolated nucleic acid molecule of claim 1, wherein the 4-1BB intracellular region comprises SEQ ID NO: 13 and the CD3ζ intracellular region comprises SEQ ID NO: 14.

9.-10. (canceled)

11. The isolated nucleic acid molecule of claim 1, wherein the CD8 hinge region comprises SEQ ID NO: 11 and the CD8 transmembrane region comprises SEQ ID NO: 12.

12. (canceled)

13. The isolated nucleic acid molecule of claim 1, wherein the nucleic acid further comprises a peptide linker 3 to 10 amino acid residues in length linking the antibody and the CD8 hinge region.

14.-20. (canceled)

21. The isolated nucleic acid molecule of claim 1, wherein the IL-7 comprises SEQ ID NO: 18.

22. The isolated nucleic acid molecule of claim 1, wherein the CCL19 comprises SEQ ID NO: 19.

23. (canceled)

24. The isolated nucleic acid molecule of claim 1, wherein the isolated nucleic acid molecule encodes a polypeptide comprising SEQ ID NO: 16.

25. The isolated nucleic acid molecule of claim 1, wherein the isolated nucleic acid molecule comprises SEQ ID NO: 17.

26. A vector comprising the nucleic acid molecule of claim 1.

27.-31. (canceled)

32. An immune cell derived from a mammal or separated from a mammal and expressing a) a chimeric antigen receptor (CAR) comprising an antibody that specifically recognizes human mesothelin, a CD8 hinge region, a CD8 transmembrane region, a 4-1BB intracellular region and a CD3ζ intracellular region, b) IL-7, and c) CCL19.

33. (canceled)

34. A pharmaceutical composition comprising the immune cell of claim 32 and a pharmaceutically acceptable additive.

35. A method of treating a mesothelin-expressing cancer comprising administering to a subject in need thereof the pharmaceutical composition of claim 34.

36.-40. (canceled)

41. The method of claim 35, wherein the method further comprises administering to the subject an additional therapeutic agent or an additional therapeutic regimen.

42.-49. (canceled)

50. A method of decreasing tumor cell proliferation comprising contacting the tumor cell with the immune cell of claim 32, thereby decreasing the tumor cell proliferation.

51.-52. (canceled)

53. A method for producing an immune cell expressing cell surface molecules that specifically recognize human mesothelin, IL-7, and CCL19, the method comprising:
introducing the nucleic acid molecule of claim 1 to an immune cell to induce expression of cell surface molecules that specifically recognize human mesothelin, IL-7, and CCL19 by the immune cell.

54. (canceled)

55. A kit comprising the nucleic acid molecule of claim 1 and instructions of use.
